(19) United States
(12) Patent Application Publication
(10) Pub. No.: US 2025/0387073 A1
(43) Pub. Date: Dec. 25, 2025

(54) METHOD AND SYSTEM FOR NON-INVASIVE PREDICTION OF TISSUE COMPOSITION FROM MRI AND BLOOD-BASED BIOMARKERS

(71) Applicant: The University of Chicago, Chicago, IL (US)

(72) Inventors: Mustafa Batuhan Gundogdu, Chicago, IL (US); Aytekin Oto, Chicago, IL (US); Gregory S. Karczmar, Crete, IL (US); Aritrick Chatterjee, Chicago, IL (US); Milica Medved, Chicago, IL (US)

(21) Appl. No.: 19/244,195

(22) Filed: Jun. 20, 2025

Related U.S. Application Data
(60) Provisional application No. 63/662,531, filed on Jun. 21, 2024.
(60) Provisional application No. 63/708,965, filed on Oct. 18, 2024.

(51) Int. Cl. A61B 5/00; A61B 5/055; G01R 33/50; G01R 33/563

(52) U.S. Cl. CPC A61B 5/4381; A61B 5/004; A61B 5/055; G01R 33/50; G01R 33/56341

(57) ABSTRACT

A system for assessing cancer includes a memory configured to store one or more images of tissue of a patient, where the one or more images originate from a magnetic resonance imaging (MRI) machine. The system also includes a processor operatively coupled to the memory and configured to determine a composition of the tissue. The processor is also configured to determine, based at least in part on the composition of the tissue, an apparent diffusion constant of the tissue. The processor is also configured to identify a region of cancer based at least in part on the apparent diffusion constant. The processor is further configured to generate a map that identifies the region of cancer in the tissue.

### CROSS-REFERENCE TO RELATED APPLICATIONS

The present application claims the priority benefit of U.S. Provisional Patent App. No. 63/662,531 filed on Jun. 21, 2024 and U.S. Provisional Patent App. No. 63/708,965 filed on Oct. 18, 2024, both of which are incorporated by reference herein in their entirety.

### REFERENCE TO GOVERNMENT RIGHTS

This invention was made with government support under CA228036 awarded by the National Institutes of Health. The government has certain rights in the invention.

### BACKGROUND

Prostate cancer is one of the most common types of cancer among men. Prostate cancer is the fourth most commonly diagnosed cancer in the world, and is believed to be diagnosed approximately 1.5 million times every year. Diagnosis and treatment of prostate and other forms of cancer is often dependent on tissue imaging techniques, such as magnetic resonance imaging (MRI). Given the need for fast, accurate imaging in cancer diagnosis and treatment, it is important for the imaging system being used to be able to efficiently deal with noise and signal degradation during the imaging process.

### SUMMARY

An illustrative system for assessing cancer includes a memory configured to store one or more images of tissue of a patient, where the one or more images originate from a magnetic resonance imaging (MRI) machine. The system also includes a processor operatively coupled to the memory and configured to determine a composition of the tissue. The processor is also configured to determine, based at least in part on the composition of the tissue, an apparent diffusion constant of the tissue. The processor is also configured to identify a region of cancer based at least in part on the apparent diffusion constant. The processor is further configured to generate a map that identifies the region of cancer in the tissue.

In an illustrative embodiment, the composition of the tissue includes a fractional volume of stroma in the prostate tissue. In another embodiment, the composition of the prostate tissue includes a fractional volume of lumen in the tissue. In another embodiment, the composition of the prostate tissue includes a fractional volume of epithelium in the tissue. In one embodiment, the one or more images comprise a plurality of voxels, and the processor applies a 3-compartment diffusion-relaxation signal model to each voxel in the plurality of voxels.

In another embodiment, the processor determines, based on the composition of the tissue, one or more relaxation times (T2) of the tissue, and the region of cancer is identified based at least in part on the one or more relaxation times. In another embodiment, the processor determines, based on the composition of the tissue, a volume of the tissue, and wherein the region of cancer is identified based at least in part on the volume of the tissue. In one embodiment, the processor uses an encoder to determine the apparent diffusion constant of the tissue.

In an illustrative embodiment, the apparent diffusion constant is determined with respect to an epithelium portion of the tissue, a lumen portion of the tissue, and a stroma portion of the tissue. In another embodiment, the processor determines an echo time and a b-value of the tissue from the one or more images, and the composition of the tissue is determined based at least in part on the echo time and the b-value. In one embodiment, the tissue comprises prostate tissue and the processor is configured to determine a normalized prostate specific antigen (PSA) density of the prostate tissue, and the region of cancer is identified based on the normalized PSA density, which acts as a biomarker. In another embodiment, the tissue comprises prostate tissue and the processor determines a first normalized PSA density of an epithelial portion of the prostate tissue and a second normalized PSA density of a lumen portion of the prostate tissue. In another embodiment, the processor determines the normalized PSA density based on prostate volume, tissue volumes within the prostate, and a PSA density of the prostate tissue.

An illustrative method of assessing cancer risk includes receiving, by a memory of a computing system, one or more images of tissue, where the one or more images originate from a magnetic resonance imaging (MRI) machine. The method includes determining, by a processor of the computing system, a composition of the tissue. The method also includes determining, by the processor and based at least in part on the composition of the tissue, an apparent diffusion constant of the tissue. The method also includes identifying, by the processor, a region of cancer based at least in part on the apparent diffusion constant. The method further includes generating, by the processor, a map that identifies the region of cancer in the tissue.

In one embodiment, determining the composition of the tissue includes determining a fractional volume of stroma in the tissue, determining a fractional volume of lumen in the tissue, and determining a fractional volume of epithelium in the tissue. In another embodiment the method includes determining, by the processor and based on the composition of the tissue, one or more relaxation times (T2) of the tissue, where the region of cancer is identified based at least in part on the one or more relaxation times. In another embodiment, determining the apparent diffusion constant comprises determining a first apparent diffusion constant with respect to an epithelium portion of the tissue, determining a second apparent diffusion constant with respect to a lumen portion of the tissue, and determining a third apparent diffusion constant with respect to a stroma portion of the tissue.

In one embodiment, the method includes determining, by the processor, an echo time and a b-value of the tissue from the one or more images, where the composition of the tissue is determined based at least in part on the echo time and the b-value. In another embodiment, the tissue comprises prostate tissue and the method includes determining, by the processor a normalized prostate specific antigen (PSA) density of the prostate tissue, and wherein the region of cancer is identified based on the normalized PSA density, which acts as a biomarker. In another embodiment, the processor determines the normalized PSA density based on prostate volume, tissue volumes within the prostate, and a PSA density of the prostate tissue.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 is a table that includes a comparative analysis of biophysiologic compartmental models and other methods for prostate tissue profiling from MRI in accordance with an illustrative embodiment.

FIG. 2 is a flowchart showing a training phase and an inference phase of the proposed physics-informed autoencoder (PIA) for prostate tissue microstructure analysis in accordance with an illustrative embodiment.

FIG. 3 is a table that presents all results from in silico experiments conducted at a Signal to Noise Ratio (SNR) of 20:1 in accordance with an illustrative embodiment.

FIG. 4 shows the Mean Absolute Error (MAE) of PIA and non-linear least squares (NLLS) methods on the parameters (volume, diffusivity, T2) of the epithelium compartment as a function of test set SNR in accordance with an illustrative embodiment.

FIG. 5 displays scatterplots of true versus predicted measurements for the epithelium compartment, contrasting PIA versus NLLS methods in accordance with an illustrative embodiment.

FIG. 6 shows representative images in a patient from the cohort used in histologic analysis and the accompanying PIA analysis in accordance with an illustrative embodiment.

FIG. 7 depicts scatterplots that directly compare the prostatic tissue volume estimates from PIA against the reference standard provided by quantitative histology in accordance with an illustrative embodiment.

FIG. 8 is a table that presents a comprehensive histologic validation of PIA, alongside a comparative analysis with the conventional NLLS-based solution across various metrics in accordance with an illustrative embodiment.

FIG. 9 is a table that depicts resulting P values of the t tests conducted to assess the statistical significance of differences in epithelium diffusivity measurements between Gleason score groups in accordance with an illustrative embodiment.

FIG. 10 depicts box plots comparing epithelium measurements from both PIA and NLLS-based approaches in accordance with an illustrative embodiment.

FIG. 11 depicts in silico scatter plots of the NLLS method at 20:1 SNR in accordance with an illustrative embodiment.

FIG. 12 depicts in silico scatter plots of the PIA method at 20:1 SNR in accordance with an illustrative embodiment.

FIG. 13 depicts the change in performance for the two methods, NLLS (squares) vs PIA (circles), on all tissue parameters (vol, D, T2) of the epithelium compartment, as a function of SNR in accordance with an illustrative embodiment.

FIG. 14 depicts the change in performance for the two methods, NLLS (squares) vs PIA (circles), on all tissue parameters (vol, D, T2) of the stroma compartment, as a function of SNR in accordance with an illustrative embodiment.

FIG. 15 depicts the change in performance for the two methods, NLLS (squares) vs PIA (circles), on all tissue parameters (vol, D, T2) of the lumen compartment, as a function of SNR in accordance with an illustrative embodiment.

FIG. 16 is a table that includes details for all parameter ranges considered in the experiments in accordance with an illustrative embodiment.

FIG. 17 is a table that presents the Pearson Correlation coefficients of the tissue parameter measurements of PIA and NLLS methods, with respect to cancer aggressiveness, measured with the Gleason Score in accordance with an illustrative embodiment.

FIG. 18 is an overview of hybrid multidimensional MRI (HM-MRI) for predicting cancer risk in accordance with an illustrative embodiment.

FIG. 19 shows an embodiment of a physics-informed autoencoder for predicting cancer risk in accordance with an illustrative embodiment.

FIG. 20 depicts operations performed by an experimental setup of the proposed system in accordance with an illustrative embodiment.

FIG. 21 depicts additional operations performed by an experimental setup of the proposed system in accordance with an illustrative embodiment.

FIG. 22 depicts an epithelium volume obtained by HM-MRI, an epithelium volume map obtained by PIA, and an epithelium ADC map obtained by PIA in accordance with an illustrative embodiment.

FIG. 23 depicts another example in which a cancer was missed by HM-MRI but identified using PIA in accordance with an illustrative embodiment.

FIG. 24 depicts operations used to calculate tissue normalized PSA density in accordance with an illustrative embodiment.

FIG. 25 depicts results of a patient with PSA of 10.1 ng/ml and prostate volume of 68.1 cc with Gleason 3+4 cancer in the right mid lateral region of the prostate in accordance with an illustrative embodiment.

FIG. 26 depicts a patient with PSA of 11.1 ng/ml and prostate volume of 96.8 cc with no cancer in accordance with an illustrative embodiment.

FIG. 27 depicts a computing system for performing image analysis and cancer risk assessment in accordance with an illustrative embodiment.

### DETAILED DESCRIPTION

Tissue estimates obtained via least-squares fits to compartmental models in microstructure imaging techniques, such as hybrid multidimensional (HM)-MRI, provide a non-invasive tool for prostate cancer diagnosis, and other types of cancer diagnosis. However, the accuracy of this sum-of-exponentials-based compartmental model fit is affected by increased noise or signal degradation due to motion. Described herein is a deep learning-based approach for HM-MRI that is fast, effective, and noise-robust.

The inventors have redesigned the HM-MRI solution as a deep learning problem via physics-informed autoencoders (PIA). Instead of solving the sum-of-exponentials least-squares fit to estimate the epithelium, stroma, and lumen parameters (volume fractions, T2 and ADC for each compartment), and therefore treating them as unknowns of an equation set, the inventors model them as latent variables of an autoencoder. PIA has two parts: an encoder and a decoder. The encoder, a multi-head deep neural network, yields the parameter estimates for each compartment. These parameter estimates are (1) the volume fractions, (2) ADC and (3) the T2 of each compartment. The decoder is set nontrainable, calling the analytical function that generates theoretical MRI signal values at the output. PIA is trained in an unsupervised fashion to minimize the squared error between its noisy input and “physics-informed” output. Evaluations were conducted via both Monte Carlo simulations under various noise conditions as well as in-vivo scans of 21 patients with prostate cancer who underwent prostatectomy after imaging.

PIA provides a non-invasive and quantitative tool for detecting tissue composition, as well as the ADC and T2 behavior of each compartment. The ADC and T2 values of individual compartments, especially epithelium and stroma, correlate significantly with the aggressiveness of the cancer as measured by the Gleason score—which is not the case with the conventional model fitting-based (NLLS-based) HM-MRI. These new parameters can also be used to increase diagnostic accuracy. The tissue composition estimates of PIA can be used to potentially reduce the number of unnecessary biopsies.

PIA provides a new paradigm to the solution that the hypothesis-driven HM-MRI offers. Both approaches aim to introduce a quantitative solution to prostate MRI. In other words, both approaches aim to translate the otherwise non-quantitative signal intensities into quantitative and explainable biomarkers. Other similar technologies such as VERDICT and restricted diffusion imaging, which rely primarily on diffusion-weighted MRI to detect changes in cell density and architecture associated with cancer, and Luminal water imaging, which detects decreased luminal fluid associated with invasion of cancer into prostatic ducts, also employ hypothesis-driven solutions based on function fitting to a non-linear signal decay model. These methods however do not take into account the very strong interactions between apparent diffusion coefficient and T2 measurements. As discussed herein, PIA differs from these models as the solution is not provided by an optimization problem with a search in the parameter space, rather—from intermediate layer outputs (latent variables) of a deep neural network, which is trained to solve for the underlying tissue composition, even if the MRI acquisitions suffer from physiological noise that makes the hypothesis-driven function-fitting-based solutions (introduced above) impractical and incorrect.

This technology offers significant improvements over existing methods in several aspects. Firstly, it addresses the issue of thermal and physiological noise present in MR images used in HM-MRI and other similar technologies. Other methods that rely on minimum-error fits to sum-of-multi-exponential models for each voxel often fail to accurately measure the ADC and T2 values of tissue composition, which are important diagnostic parameters. These methods result in high errors in volume fraction estimation under increased noise. In contrast, the proposed technology takes into account the underlying noise behavior. It achieves this by training a multi-head neural network that can provide tissue composition parameters even under increased noise or when certain signals are missing.

Another advantage of this method is that it does not require supervised training with true volume fractions or ADC/T2 values as labels. Such an approach would necessitate a large dataset, which is often not practically available. Moreover, supervised models would be specific to a particular domain, meaning they may not generalize well to different imaging parameters (e.g., b-value or TE) or when working with various vendors or body parts. In contrast, this technology is physics-informed and unsupervised. The squared error at the output of the decoder guides the encoder to generate better estimates, even when the signal is unreliable.

Furthermore, once the model is trained with simulated or real data points, the real-time execution is significantly faster than the relevant technology. The preliminary experiments showed a 10,000 times faster execution, which can make it possible for radiologists to observe the predicted tissue compositions as they are examining the MRI images (0.18 seconds vs 40 minutes per image). For hypothesis-driven methods there was no such option.

Based on the above-described analysis, it was found that PIA has significantly higher correlation with the true tissue compartments and more accurate tissue estimates; compared to the least-squares-based HM-MRI solution under increased noise (0.81 vs 0.61, 0.74 vs 0.53, 0.97 vs 0.91, p<0.01, for epithelium, stroma, and lumen volume fractions, respectively) while providing about 10000× speed improvement. On in-vivo images, PIA accurately predicts increased epithelium and decreased lumen on cancer regions and is consistent with biopsy results. As such, PIA can be used for non-invasive prediction of tissue composition of the prostate from MRI and potentially as a quantitative MRI method.

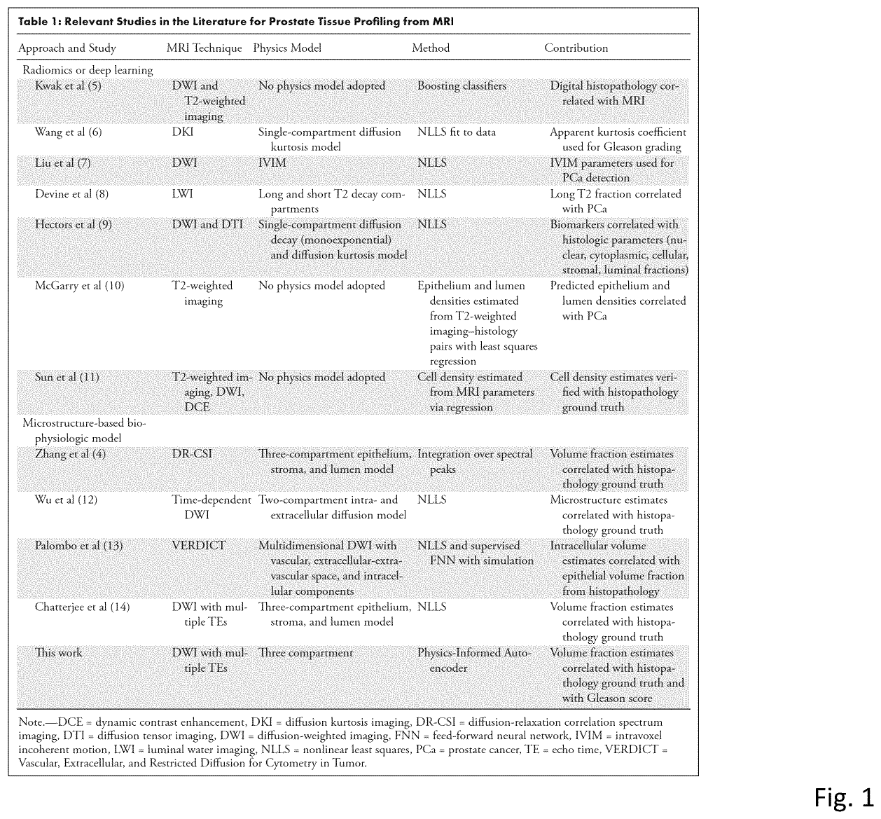

Prostate cancer (PCa) is alarmingly common, with one in eight male individuals in the United States diagnosed with PCa at some point in their lives. Multiparametric MRI with the Prostate Imaging Reporting and Data System (PI-RADS) is considered a standard of care for screening and differential diagnosis of PCa. However, the positive predictive value of PI-RADS version 2.1 is as low as 35%, leading to approximately 1 million unnecessary biopsies each year in the United States alone and causing undue stress to patients. In addition, 29% of clinically significant cancers are missed. To address the problem of the subjectivity of PI-RADS and to increase diagnostic accuracy, various researchers have turned to biophysiologic compartmental models for noninvasive inference of tissue microstructure. The overall approach with these biophysiologic compartmental models involves fitting the MRI data to a predefined function. These functions, typically a sum of decaying exponentials, represent a hypothesis about the underlying signal behavior. FIG. 1 is a table that includes a comparative analysis of biophysiologic compartmental models and other methods for prostate tissue profiling from MRI in accordance with an illustrative embodiment. While many of the examples herein relate to prostate cancer, it is to be understood that the methods and systems described herein are not limited to diagnosis of prostate cancer. The proposed methods and systems can also be used to diagnose cancer in other types of tissue, such as breast tissue, pancreatic tissue, etc. In addition, the proposed methods and systems can be used to diagnose other pathologies in addition to cancer, such as cirrhosis, radiation necrosis, etc.

A primary challenge associated with multicompartment models is that using the sum of decaying exponentials often leads to ill-posed behavior with NLLS algorithms, causing difficulties in parameter estimations. A particular concern is when various tissue compartments exhibit similar MRI decay characteristics. In such cases, the parameter estimation process becomes highly sensitive to initial guesses and noise in the data, leading to a vast solution space. This ambiguity in parameter estimation can substantially degrade the reliability of the model, as small variations in the input data can result in large changes in the estimated parameters, especially in the presence of high levels of noise.

There is a growing trend in research exploring the use of supervised deep learning for PCa detection. However, these models require large amounts of well-labeled training data, and their effectiveness across different MRI vendors remains a concern due to domain discrepancies. Physics-informed deep learning aims to integrate physical laws into neural network training, thereby facilitating solution development and avoiding the need for large training datasets. Early applications of this method focused on partial differential equations. This approach has been adapted for multiexponential signal models, such as diffusion-relaxation models of white matter microstructure and biexponential intravoxel incoherent motion models.

The current work presents an emerging self-supervised deep learning approach that bridges the gap between hypothesis-driven and data-driven methods for MRI signal analysis. The proposed method leverages the strengths of both paradigms, mitigating their inherent limitations and capitalizing on their complementary advantages. Specifically, the proposed model, Physics-Informed Autoencoder (PIA), encodes the underlying biophysical principles as a prior knowledge constraint within a neural network architecture. This innovation eliminates the need for extensive training on large datasets, a major bottleneck in conventional deep learning approaches. The purpose of this study was to evaluate the performance of PIA in measuring tissue-based biomarkers of PCa using hybrid multidimensional MRI (HM-MRI). The efficacy of the proposed method is comprehensively evaluated through in silico and in vivo experiments, where histopathologic measurements of the true tissue parameters serve as the reference standard for validation.

Materials and methods are described below. A retrospective study was conducted between June 2022 and July 2024, and presents a self-supervised deep learning approach for estimating MRI biomarkers for PCa, with histopathologic confirmation of its measurements. The framework, PIA, integrates biophysical model-based parameter fitting with deep learning methods. The first set of experiments involves development and analysis of PIA with in silico data, whereas the second set of experiments presents evaluation of PIA's performance in clinical in vivo prostate MRI scans. This study involved retrospective analysis of prospectively collected data.

The inventors developed PIA with a histologically verifiable three-compartment diffusion-relaxation model that includes three tissue compartments that include the epithelium (ep), stroma (st), and lumen (lu). In this model, a signal in each compartment decays as the b value and echo time (TE) increase, at a rate proportional to their volume fractions (vₙ). Rates are also related to the individual diffusivities (Dₙ), and T2 relaxation times (T2ₙ) such that:

$$\frac{{s}_{b , TE}}{{s}_{0 , 0}} = {\sum }_{n ∈ \{ e p , st , lu \}} {v}_{n} \times exp ( - {D}_{n} \times b - \frac{TE}{T {2}_{n}} ) . \qquad Eq . 1$$

The current state-of-the-art implementations of this method aims to infer the tissue parameters (vₙ, Dₙ, T2ₙ, n∈{ep,st,lu}) by fitting HM-MRI data, scanned with various b-value and TE pairs, to the Equation, using the NLLS optimization. Predicted parameters in Equation 1 are vₙ, Dₙ, and T2ₙ. Previous research has demonstrated that tissue volume fraction estimates derived from fitting HM-MRI data to this model using NLLS are valuable biomarkers for cancer detection. However, the exploration of diffusivity and T2 measurements within each compartment was not feasible in these studies. This was primarily due to the complexities introduced by the sum of multiexponentials, especially for images with low signal-to-noise ratio (SNR), which hinders accurate estimation of diffusivity and T2 values.

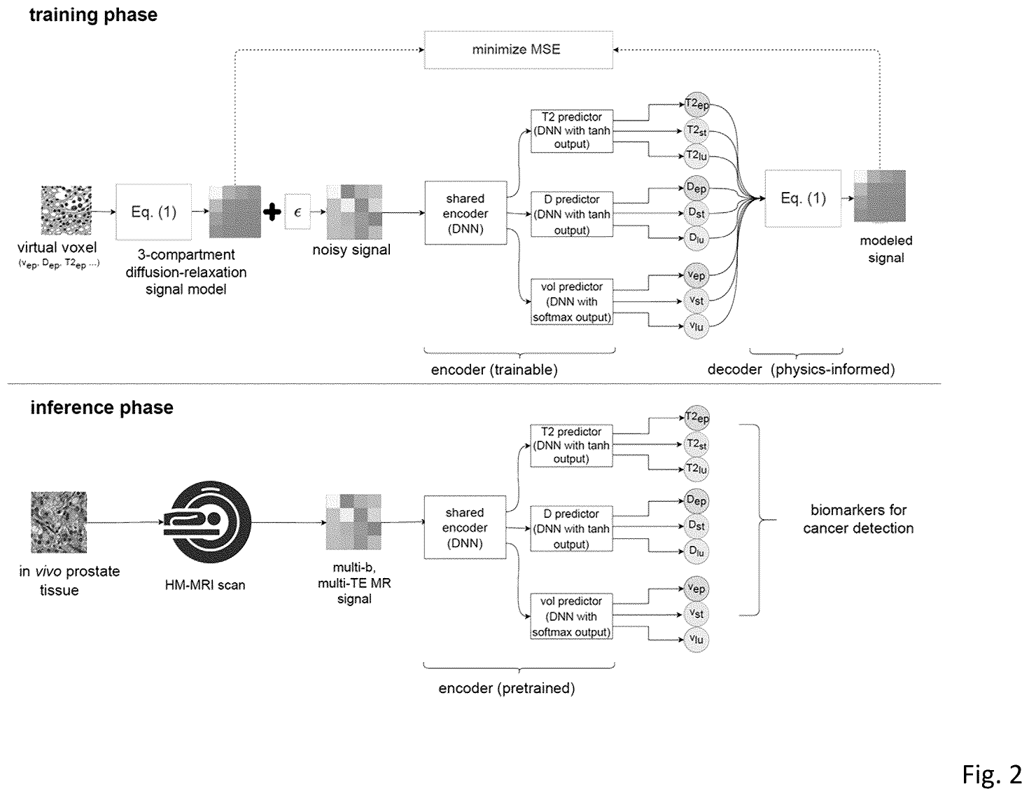

Physics-Informed Autoencoder. Traditional model-based methods treat the tissue-specific biomarkers in Equation 1 as unknowns in equations, while the proposed solution PIA transforms the problem into a deep learning task and views them as latent variables within an autoencoder. Like all other autoencoders, PIA includes two parts, an encoder and a decoder. The encoder is a trainable neural network that predicts the underlying tissue-specific biomarkers from the given MRI measurements in its input. On the other hand, the decoder is a nontrainable biophysical model function that reproduces the MRI signals using the output of the encoder. FIG. 2 is a flowchart showing a training phase and an inference phase of the proposed physics-informed autoencoder (PIA) for prostate tissue microstructure analysis in accordance with an illustrative embodiment. In FIG. 2, D=diffusivity, DNN=deep neural network, ep=epithelium, Eq.=Equation 1, HM-MRI=hybrid multidimensional MRI, lu=lumen, MSE=mean squared error, st=stroma, TE=echo time, and v=volume fraction. During training, the encoder learns to emulate the decoder's physical rules, hence the term physics-informed. PIA's encoder is a feed-forward multihead neural network.

Multiparametric MRI signals are processed by a six-layer deep neural network with leaky ReLU activations. This shared network extracts the embedding to infer the underlying biomarkers. The embedding is then processed by parameter-specific layers. The volume fraction estimation is a simple classification network with two layers and a softmax activation function. The diffusivity and T2 estimators are modeled with tanh activation functions. This design allows the PIA encoder to predict the diffusivity and T2 of each tissue compartment within their range of realistic values. The outputs of the encoder are fed to the decoder, which is the three-compartment signal model in Equation 1, to synthesize an approximation of the input MRI signal.

A core innovation of the PIA lies in its mechanism for enforcing biophysically meaningful constraints on inferred biomarkers—such as diffusivities and relaxation times—through carefully constructed nonlinearities applied at the encoder's output. This strategy replaces the crude and often non-differentiable cutoffs used in conventional approaches with smooth, continuous functions that inherently respect physical limits. For biomarkers appearing in the exponents of the three-compartment tissue signal model (e.g., the diffusivity or T₂ of epithelial tissue), PIA applies a scaled and shifted hyperbolic tangent (tanh) function. This transformation maps the unbounded output of the encoder to a specified physical range [Dmin,Dmax]. For instance, the diffusivity estimate for a given compartment can be computed as:

$$\hat{D} = \frac{( {D}_{max} - {D}_{min} ) \times tanh ( encoder ( x ) ) + ( {D}_{max} + {D}_{min} )}{2} ,$$

where x represents the observed MRI signal and encoder( ) is the trainable deep neural network encoder of PIA.

This construction has several important advantages: 1. The use of the tanh ensures that the output remains strictly within the physically feasible interval. As the encoder output tends toward ±∞, the tanh asymptotically approaches ±1, and the final estimate correspondingly approaches Dₘᵢₙ or Dₘₐₓ, never exceeding them. This makes the bounds intrinsic to the model rather than imposed post hoc. 2. More importantly, at early stages of the training, if the output becomes too big of a number or too small of a number, it can always propagate to a better (lower error) intermediate value since the scaled tanh function is differentiable everywhere, as opposed to the application of hard cutoffs as used in other solutions.

A similar approach is used to estimate tissue volume fractions, which must satisfy both positivity and unit-sum constraints. Here, PIA leverages the softmax function which interprets the volumes as a probability mass function over tissue compartments. This formulation guarantees that all estimated fractions are in the interval (0, 1) and that their sum is exactly 1. Moreover, like the tanh, the softmax is smooth and differentiable, facilitating stable training dynamics and allowing small errors to be corrected efficiently during backpropagation. Together, these design choices embed biophysical priors directly into the model architecture, enabling PIA to learn representations that are not only accurate but also physically plausible, even in data-scarce or noisy regimes.

Training Method. The PIA was trained in a self-supervised fashion. The objective of the pretraining phase was to have the encoder learn to emulate the inverse of the biophysical model, especially under adverse noise conditions. The training dataset for the pretraining phase included synthetically generated data with various tissue compositions of epithelium, stroma, and lumen compartments. The compartment parameters (vₙ, Dₙ, T2ₙ, n∈{ep,st,lu}) were sampled uniformly from within biophysically realistic parameter ranges for each compartment, for example, Dₛₜ in range 0.7-1.7 micrometer squared per second (μm²/sec). It is noted that the sampled tissue values were never used to supervise PIA; instead, the inventors generated synthetic MRI signals by applying the biophysical model (Equation 1).

To establish robustness to noise, the virtual MRI signals were corrupted with normally distributed additive noise at both real and imaginary components, and the magnitude of the noisy signal was used as input to PIA. This procedure made the input magnitude data Rician-distributed, as is the case for in vivo data. The standard deviation (SD) of the additive noise was set so that the SNR of the maximum signal amplitude (lowest echo time, lowest b-value signal amplitude) was 20:1. The inventors trained PIA with an SNR of 20:1 because it is reported to be the expected SNR found in prostate tissue.

In the forward run, the encoder predicts the underlying tissue parameters from the noise-corrupted signal and the decoder reconstructs the MRI signal based on the encoder's estimates. The model is trained to minimize the squared error between the reconstructed signal and the noise-free version of the input signal. At every epoch, a new batch of virtual data, each with different random noise and a set of tissue parameters, was generated with random sampling. The training was kept for 50 000 epochs. A learning rate of 0.0003 was used with Adam optimizer. The hyperparameters of the PIA model, including the encoder complexity, learning rate, and length, were set so that the multiheaded encoder could “memorize” the inverse of Equation 1 under noise-free scenarios. This was established in a hyperparameter tuning phase by measuring the reconstruction error prior to the training with added noise.

In Silico Validation. During inference, the encoder outputs are taken as the tissue parameter estimates of PIA. First, PIA's performance in estimating the underlying parameters was evaluated using the reference standard volume, diffusivity, and T2 parameters under several conditions to test for robustness to MRI variations: (a) under various noise conditions with SNR levels between 10:1 and 10 000:1, (b) under a different imaging protocol (train with endorectal coil MRI protocol and test with surface coil MRI protocol), and (c) under a different tissue model (train with three-compartment model and test with two-compartment data). Furthermore, the inventors investigated PIA's performance under conditions in which epithelium and stroma exhibit similar MRI decay characteristics, a situation where NLLS is known to fail. Evaluations were conducted using the following metrics: (a) Spearman correlation coefficient, (b) mean absolute error (MAE), (c) bias, and (d) SD.

To assess the speed performance of PIA in comparison to NLLS, both methods were executed on the same set of 20 000 virtual voxels and the wall time for their solution was measured on an Intel Xeon Gold 6130 central processing unit (CPU) with 2.10 GHz. In alternative embodiments, different computing hardware may be used.

Histologic Validation with in Vivo Scans. The inventors validated the performance of PIA's volume fraction estimations using histologic measurements of patients with PCa. The HM-MRI scans from 21 patients with PCa who underwent prostatectomy after imaging (mean age, 60 years 6.6 [SD]; all male) were examined. This cohort has been previously used in a published work for the histologic validation of HM-MRI using the NLLS method. Here it is used to validate the in vivo accuracy of PIA biomarkers and compare them to NLLS and quantitative histology. A total of 71 regions of interest (ROIs), comprising 35 cancerous and 36 healthy tissues from the 21 patients, were evaluated for tissue compartment percentages, using quantitative histology as the benchmark.

Agreement between PIA's volume fraction estimations and histologic measurements was assessed using the intraclass correlation coefficient (ICC). Performance of PIA's volume fraction estimations for estimating histologic measurements was evaluated using linear mixed modeling, with PIA volume fraction as the fixed effect and the subjects as random effects. The marginal (unconditional) R2 value was used as the metric for prediction performance.

Evaluation of in Vivo Diffusivity and T2 Estimates of PIA. Quantitative histology measurements served as the reference standard for volume fraction estimates and a way to validate PIA's in vivo performance, as the inventors are not aware of a direct way to validate the performance of PIA in measuring diffusivities and T2 relaxation times of individual tissue compartments in in vivo scans of human prostates (although, in principle, MRI microscopy could provide this information for ex vivo tissues). Correlation of PIA's diffusivity and T2 measurements of tissue compartments with the Gleason grade, serving as the reference standard, was calculated using the Pearson correlation coefficient. Gleason grades were classified into five categories: healthy (n=36), 3+3 (n=9), 3+4 (n=14), 4+3 (n=9), and 4+4 and above (n=3).

Diagnostic Utility and Interpretability of PIA's Measurements. Clinical utility of epithelium and lumen volumes as biomarkers for clinically significant PCa (CSPCa) detection have been previously shown. In a receiver operating characteristic curve analysis, including CSPCa (Gleason score 3+4 and above) and benign tissues (Gleason score 3+3 and below), PIA's biomarker estimates for the 71 ROIs were compared against the estimates of NLLS and conventional apparent diffusion coefficient (ADC)-based measurements.

The interpretability of PIA biomarkers was analyzed via feature importance in CSPCa detection using the permutation importance method. A random forest model was fit on the in vivo dataset, and the importance of each biomarker was assessed by randomly shuffling its values and observing the resulting decrease in model performance. This process was repeated 10 times to obtain an average importance score for each feature to gauge their respective contributions.

Statistical Analysis. In in silico tests, when comparing PIA's biomarkers with the estimates from NLLS based on the reference standard, the inventors used Steiger Z test for Spearman r, t test for MAE and bias, and F test for the SD, using a significance P value of 0.05. When multiple tests were conducted, Bonferroni correction was applied. Consequently, the significance values were reduced to 0.00139. In in vivo tests, the metrics obtained using PIA-derived volume fraction estimations were compared with those obtained using NLLS method-derived volume fraction estimations using a one-sided Z test. The inventors used the t test for MAE and absolute bias and the F test for the SD. All standard errors for the differences were determined using the cluster bootstrap method, to account for multiple ROIs defined in each patient. The cluster bootstrap was implemented by resampling, with replacement, the patients to ensure that the correlation in outcome measures between ROIs within the patients was maintained. The cluster bootstrap procedure was iterated B=9999 times to minimize the simulation error to the extent possible. Analyses were performed in R (version 4.4.1) and Python (version 3.12.5). The significance levels were set to 0.05.

For diagnostic utility tests, the inventors used the DeLong test for area under the receiver operating characteristic curve (AUC) and performed pairwise t tests between each Gleason score group to evaluate the efficacy of PIA's epithelium diffusivity measurements in detecting PCa aggressiveness.

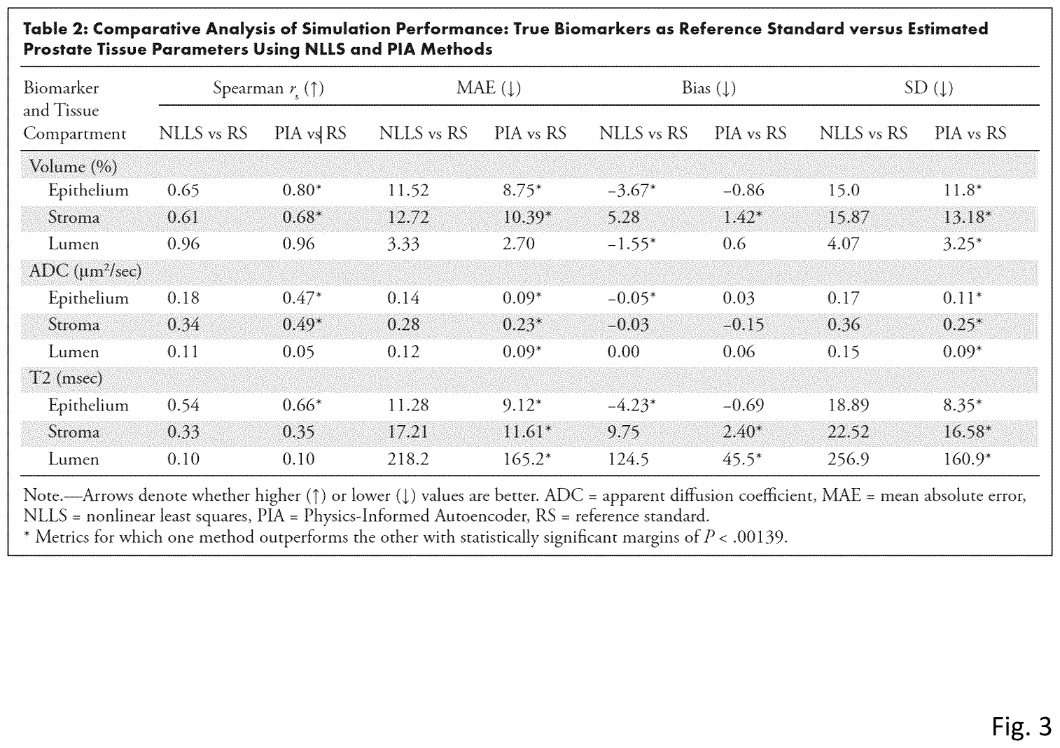

Results are discussed below. For the in Silico Experiments, PIA estimated the imaging biomarkers with superior performance over NLLS with respect to Spearman r, MAE, bias, and SD metrics. At an SNR of 20:1 and in volume of epithelium, for instance, which is the strongest biomarker for PCa detection, PIA's estimations had significantly higher correlations with the reference standard volume over NLLS (0.80 vs 0.65, P<0.001) and lower MAE (0.09 vs 0.12, P<0.001). FIG. 3 is a table that presents all results from in silico experiments conducted at an SNR of 20:1 in accordance with an illustrative embodiment.

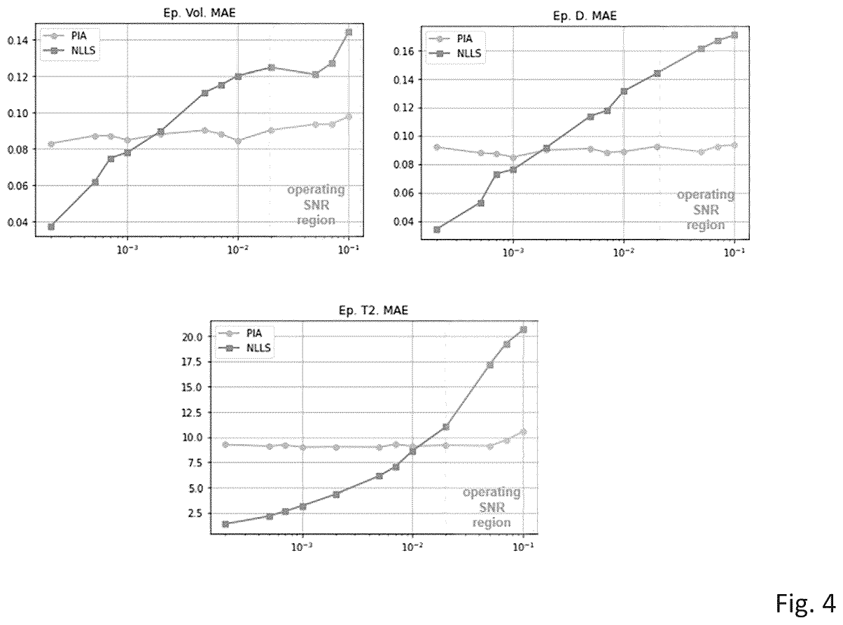

FIG. 4 shows the MAE of PIA and NLLS methods on the parameters (volume, diffusivity, T2) of the epithelium compartment as a function of test set SNR in accordance with an illustrative embodiment. More specifically, FIG. 4 shows change in mean absolute error (MAE) performance for the two methods, nonlinear least squares (NLLS) (square) versus Physics-Informed Autoencoder (PIA) (circle), on all tissue parameters (volume fraction [Vol.], diffusivity [D.], and T2) of the epithelium (Ep.) compartment, as a function of signal-to-noise ratio (SNR). As expected, NLLS yields accurate measurements under very high SNR levels. However, as noise increases to levels experienced in clinical applications of MRI, the solutions of NLLS quickly degrade (note the log scale). PIA, however, presents robustness against noise and outperforms NLLS significantly under realistic operating SNR conditions as observed in clinical MRI scans (shaded region). Thus, in an ideal scenario with no or negligible noise levels, NLLS provides the best solution. However, in more realistic operating points of an SNR of 20:1 and worse, the NLLS solution quickly degrades. PIA, on the other hand, shows robustness to noise and keeps reliable estimates.

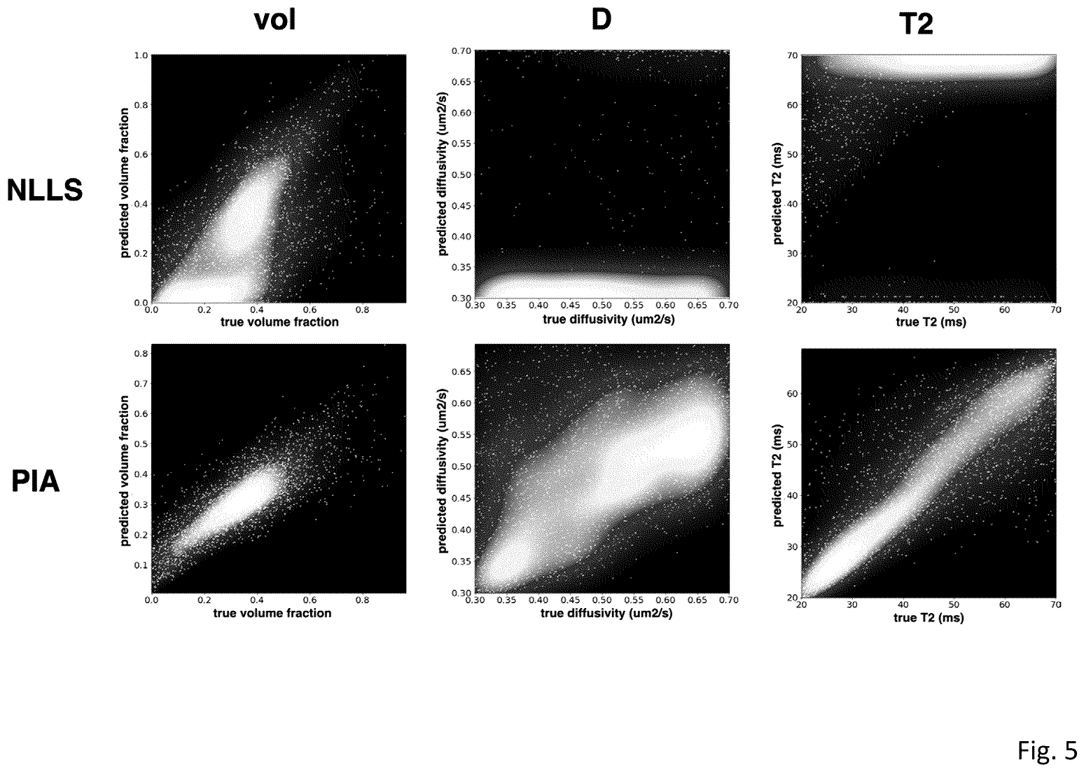

FIG. 5 displays scatterplots of true versus predicted measurements for the epithelium compartment, contrasting PIA versus non-linear least squares (NLLS) methods in accordance with an illustrative embodiment. Areas with a higher scatter point density are depicted with warmer colors to highlight the prediction performance. In FIG. 5, D=diffusivity and vol=volume fraction.

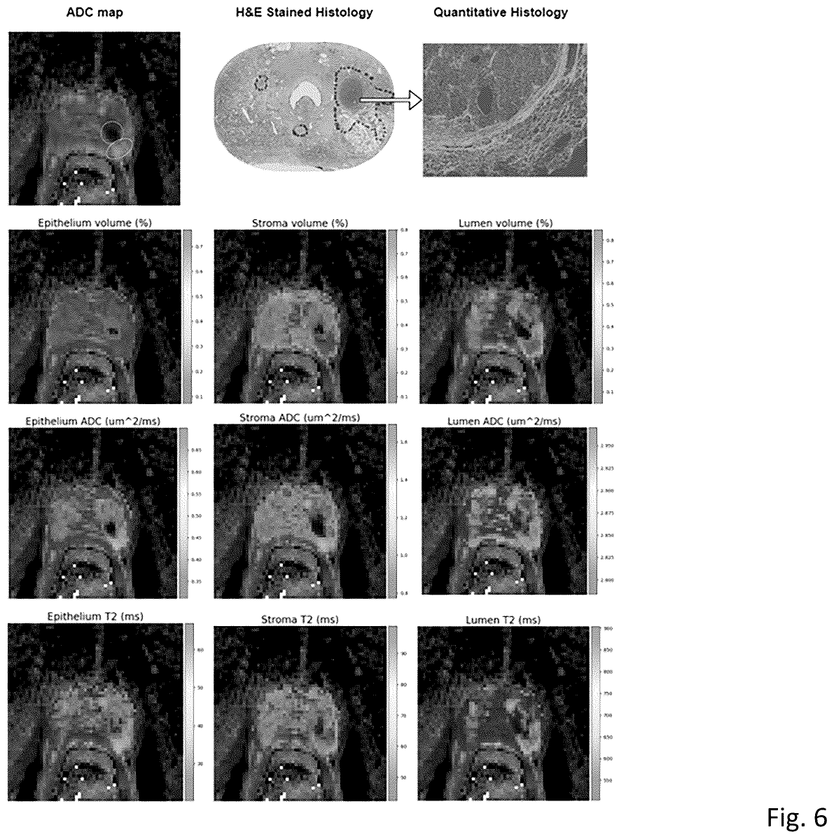

Histologic Validation. FIG. 6 shows representative images in a patient from the cohort used in histologic analysis and the accompanying PIA analysis in accordance with an illustrative embodiment. More specifically, FIG. 6 shows representative images in a 62-year-old male patient with prostate cancer that exhibit two different pathologies on the same section (cancer and cystic atopy). Top row, from left to right: Apparent diffusion coefficient (ADC) map from the axial view (non-contrast), hematoxylin-eosin (H&E)-stained histology slice with Å˜20 magnification, and image from quantitative histology of the cancer region of interest. Overlays on MR images in the second, third, and fourth rows show the Physics-Informed Autoencoder estimates of the volume fraction, ADC, and T2 of the three compartments. Epithelium volume, epithelium ADC, and stroma ADC are great indicators for cancer. Epithelium volume highlights cancer whereas the lumen volume highlights the region with cystic atrophy on the left peripheral zone.

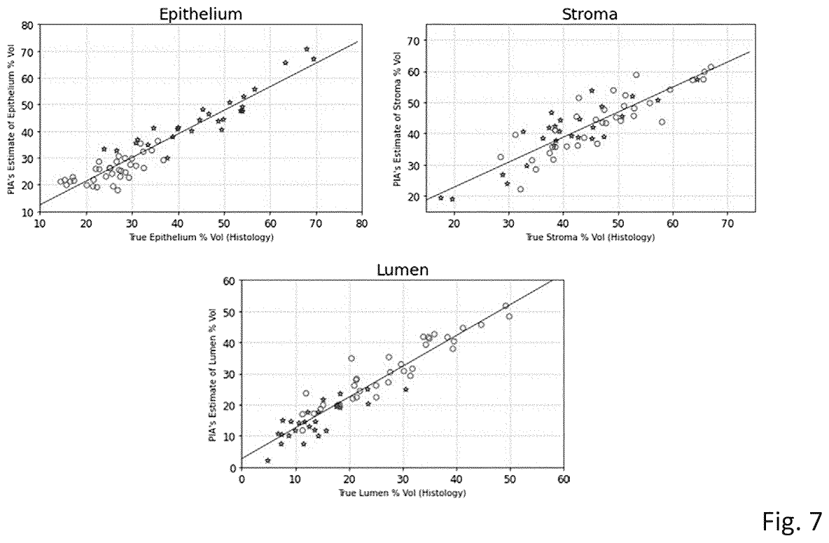

PIA's prostate tissue composition measurements demonstrated excellent agreement with quantitative histology, achieving ICC values of 0.94 (epithelium), 0.85 (stroma), and 0.92 (lumen) (P<0.001) across the three prostatic compartments. FIG. 7 visualizes this performance. Specifically, FIG. 7 depicts scatterplots that directly compare the prostatic tissue volume estimates from PIA against the reference standard provided by quantitative histology in accordance with an illustrative embodiment. In FIG. 7, X-axes show the reference standard histology volumes for each region of interest (ROI), and the y-axes show the noninvasive estimates obtained by PIA. Stars are the cancer ROIs, and circles are the benign ROIs.

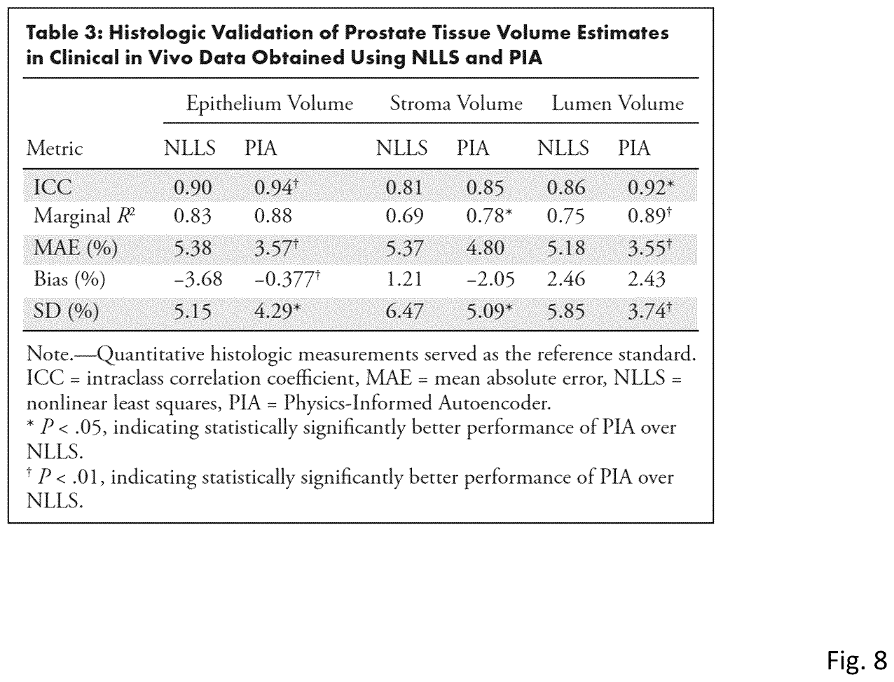

PIA's prostate tissue composition measurements also demonstrated excellent linear prediction performance with quantitative histology, achieving marginal R2 values of 0.88 (epithelium), 0.78 (stroma), and 0.89 (lumen) across the three prostatic compartments. In addition to yielding more accurate estimates on in silico experiments, PIA outperformed NLLS on in vivo evaluations as well. FIG. 8 is a table that presents a comprehensive histologic validation of PIA, alongside a comparative analysis with the conventional NLLS-based solution across various metrics in accordance with an illustrative embodiment.

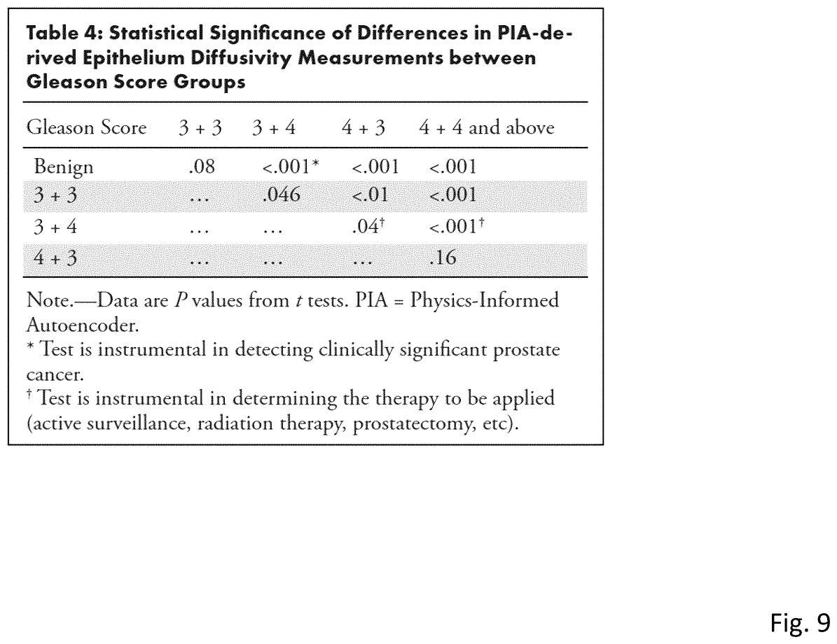

Diagnostic Utility and Interpretability of PIA's Measurements. PIA's in vivo volume fraction estimates of epithelium present a similar AUC (P=0.33) to NLLS (0.99 vs 0.97), surpassing the AUC of conventional ADC of 0.90 (P<0.02). More importantly, PIA's measurements of compartment diffusivities yielded significantly better AUC values for their utility in CSPCa differentiation than the diffusivities of NLLS (0.89 vs 0.62, P<0.001 and 0.86 vs 0.60, P<0.001 for epithelium and stroma, respectively). FIG. 9 is a table that depicts resulting P values of the t tests conducted to assess the statistical significance of differences in epithelium diffusivity measurements between Gleason score groups in accordance with an illustrative embodiment.

In silico experiments with various SNR levels demonstrated that PIA is robust in the presence of noise, especially for the exponential terms (i.e., the diffusivity and T2 of tissue compartments), whereas the NLLS method struggles with these terms when the noise levels are high (see FIGS. 4 and 5). In vivo, PIA's diffusivity estimates for epithelium and stroma demonstrated a strong inverse correlation with cancer aggressiveness, with correlation coefficients of r=−0.75 and r=−0.64, respectively. In contrast, the NLLS-based solution, as observed in in silico experiments, tended to converge to erroneous and less meaningful results for the exponential terms.

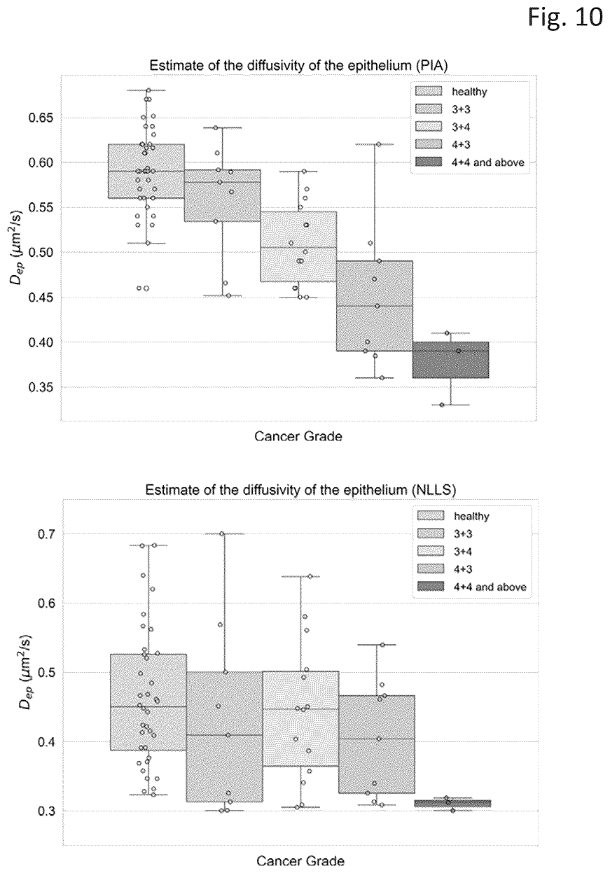

FIG. 10 depicts box plots comparing epithelium measurements from both PIA and NLLS-based approaches in accordance with an illustrative embodiment. More specifically, the box plots in FIG. 10 display PIA- and NLLS-derived epithelium diffusivity (Dep) measurements of ROIs in the patient cohort, coded with respect to each ROI's cancer aggressiveness, and measured with Gleason score. PIA's measurements of Dep (left) demonstrate a distinct inverse relationship with cancer aggressiveness, whereas the NLLSbased approach (right) does not. Each box denotes the middle 50% of the data from first quartile to third (IQR), the horizontal line in each box denotes the median, the whiskers denote data within 1.5×IQR from Q1 and Q3, and outlier points beyond the whiskers were plotted individually. Thus, in the feature importance analysis, epithelial volume fraction was a better biomarker than the conventional ADC by a large margin for CSPCa detection.

Practicality and Speed. On 20 000 virtual voxels, PIA provided a speed improvement of up to a factor of 10 000. NLLS took 2345.39 seconds (about 40 minutes) for 20 000 voxels (almost equivalent to the calculation for one MRI section), where PIA took only 0.18 second.

Thus, described above is an emerging method, PIA, that integrates the strengths of physics-based and deep learning-based methods for tissue microstructure profiling using MRI to detect and stage PCa noninvasively. The proposed solution could measure the reference standard prostate tissue volumes as validated with histopathologic measurements (ICC, 0.94, 0.85, and 0.92 for epithelial, stromal, and luminal volume fraction, respectively; P<0.001 for all), significantly outperforming NLLS in both in silico and in vivo experiments (rs, 0.80 vs 0.65; P<0.001 for epithelial volume fraction at an SNR of 20:1) and providing an accurate measurement of epithelial diffusivity as a new biomarker for measuring PCa aggressiveness (r=0.75, P<0.001).

It was shown that PIA provides noise robustness for the diffusivity and T2 estimates for the tissue compartments in in silico experiments. For in vivo analysis, there is no straightforward way to validate measured diffusivities. Thus, the inventors used correlations of PIA's measurements with PCa aggressiveness to investigate the diagnostic utility of these measures, not to prove accuracy. Nevertheless, the results showed that measurements of the diffusivity and T2 of each tissue compartment with PIA have potential to produce more effective PCa detection models and improve diagnostic accuracy.

It is acknowledged that the diffusivity distributions overlap between Gleason scores 3+3 and 3+4, as well as between 3+4 and 4+3. This overlap is expected due to the shared Gleason patterns inherent in these scoring categories. Nevertheless, the significant differences observed between benign tissue and Gleason score 3+4 indicate that PIA biomarkers can enhance the CSPCa detection. These measurements are important because the existing literature indicates that denser epithelial cells, characterized by a higher nuclear to cytoplasm ratio, are associated with more aggressive cancers.

Additionally, an increase in stiffer, diffusion-restricting fibroblasts is noted in the stroma of cancerous tissue. Hence, the findings with negative correlation of epithelium diffusivity and moderate positive correlation of epithelium T2 versus the Gleason score were reassuring and consistent with the literature. The NLLS-based method had already reported excellent correlation with histology-based tissue volumes. In addition to superior accuracy over the conventional least squares solution, the speed of the proposed model is critical in clinical contexts. The NLLS model is based on a constrained optimization algorithm, solved de novo for each new voxel. On the other hand, PIA is an analytical function modeled with neural networks, allowing orders of magnitude faster calculations. PIA's efficiency allows seamless integration with picture archiving and communications systems, enabling real-time estimation of tissue parameters during radiologic examinations. In contrast, the NLLS-based system's slower processing speed hinders its potential for real-time application.

Even with a limited sample size, the statistically significant results observed in this initial study are encouraging. The results show that PIA provides several advantages over conventional methods. First, in contrast to conventional end-to-end deep learning strategies for PCa detection, PIA reduces dependency on extensive training data and learns from physical properties of the tissue structure and imaging. This framework provides a truly explainable solution. Second, implementation of a denoising autoencoder-inspired training regimen ensures robustness against noise. Finally, the results of the study suggest that PIA can provide new information regarding tissue properties (e.g., the diffusivity and T2 of each compartment), and this can potentially improve diagnostic accuracy.

Additional details regarding implementation of the above-discussed study are included below. Parameters estimation accuracy of the proposed model was evaluated using the following metrics:

$$Pearson r = \frac{{\sum }_{n = 1}^{N} ( {y ( n )}_{pred} - \overline{{y}_{pred}} ) ( {y ( n )}_{true} - \overline{{y}_{true}} )}{\sqrt{{\sum }_{n = 1}^{N} {( {y ( n )}_{pred} - \overline{{y}_{pred}} )}^{2} {\sum }_{n = 1}^{N} {( {y ( n )}_{true} - \overline{{y}_{true}} )}^{2} )}} \qquad Eq . 2$$

$$MAE = \frac{1}{N} {\sum }_{n = 1}^{N} \vert {y ( n )}_{pred} - {y ( n )}_{true} \vert \qquad Eq . 3$$

MAE is valuable because it gives the expected error range with the units of the measurements (e.g. percentage for volumes, ms for T2, etc.) as opposed to Mean Squared Error. This metric is interpretable such that it helps to decide how much to trust each estimator.

$$Bias = \frac{1}{N} {\sum }_{n = 1}^{N} ( {y ( n )}_{pred} - {y ( n )}_{true} ) \qquad Eq . 4$$

Bias is also in the units of measurement but the sign is important to decide if one method is over- or underestimating one parameter.

$$SD = \sqrt{\frac{1}{N} {\sum }_{n = 1}^{N} {( {y ( n )}_{pred} - {y ( n )}_{true} - Bias )}^{2}} \qquad Eq . 5$$

SD is useful to evaluate the model's robustness. A model can still have low SD and high MAE if there's a high bias.

Details on MR Imaging. Patients who had previously been diagnosed with prostate cancer through histological confirmation underwent preoperative imaging using a multiparametric 3.0-Tesla MRI scanner (Achieva; Philips Healthcare, Best, the Netherlands). This process utilized a six-channel cardiac phased-array coil positioned around the pelvis in conjunction with an endorectal coil (Medrad eCoil; Bayer Healthcare, Whippany, NJ). To minimize movement of the rectal wall, a 1-mg dose of glucagon (Glucagon; Eli Lilly & Co, Indianapolis, Ind) was administered. The imaging sequence employed was a sophisticated hybrid multidimensional MRI technique, incorporating a spin-echo module with diffusion sensitizing gradients aligned symmetrically around the 180° pulse, followed by a single-shot echo-planar imaging readout. This technique enabled the capture of images at echo times of 57, 70, 150, and 200 milliseconds. For each echo time, images were taken with b values of 0, 100, 1000, and 1500 sec/mm², yielding 16 data points for each image voxel. The hybrid multidimensional MRI images were obtained in the axial view, oriented perpendicularly to the rectal wall, with the positioning guided by sagittal images. Imaging parameters were as follows: a repetition time of 3.5 seconds, a field of view measuring 180×180 mm², a slice thickness of 3 mm, and a reconstruction matrix of 128×128. The total imaging acquisition time ranged between 12 to 15 minutes.

Details on Histopathology Measurements. After their MRI scans, the patients underwent radical prostatectomy procedures. The removed prostate glands were preserved in formalin and systematically sectioned in a manner closely aligned with the MR images, then divided into quadrants. The prepared tissue sections were then embedded in paraffin, and slides stained with hematoxylin and eosin were prepared. An experienced pathologist, with over a decade of expertise, reviewed the slides for evidence of prostatic adenocarcinoma. Tumor areas were identified and marked on the histological slides. These slides were digitally scanned at 20× magnification using a whole-mount digital microscope (Olympus VS120; Olympus), and the images were saved as Olympus Virtual Slide files. The images underwent semi-automatic segmentation with the use of Image Pro Premier (Media Cybernetics), based on criteria such as color, intensity, morphology, and the exclusion of background elements, facilitated by the Smart Segment tool. The volumes of the gland components were quantified as percentages of the total image area using the “Count” tool.

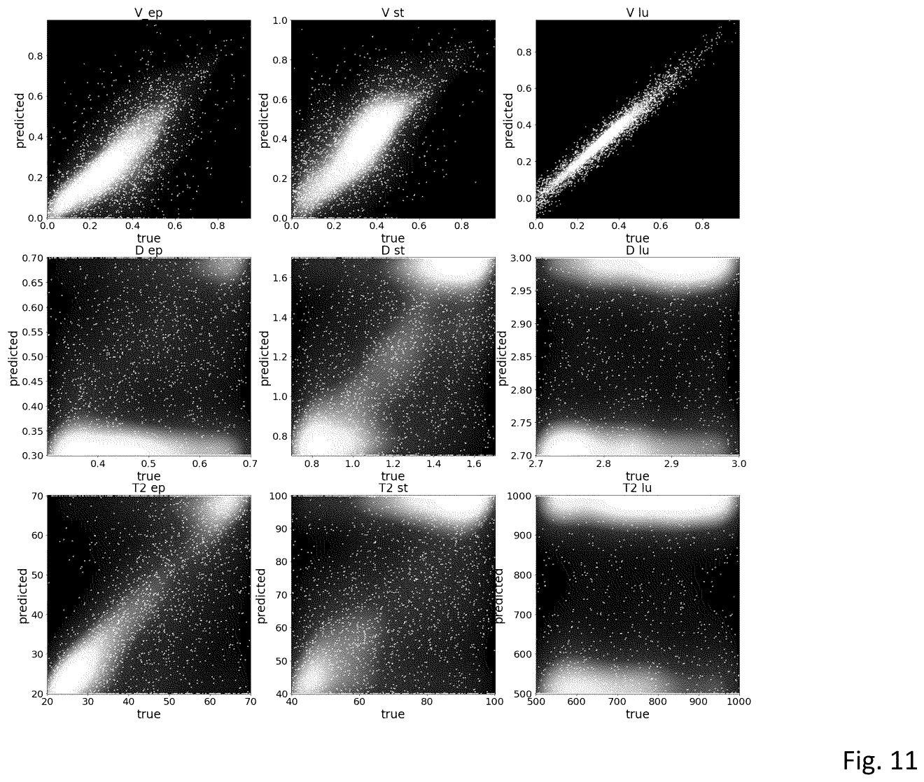

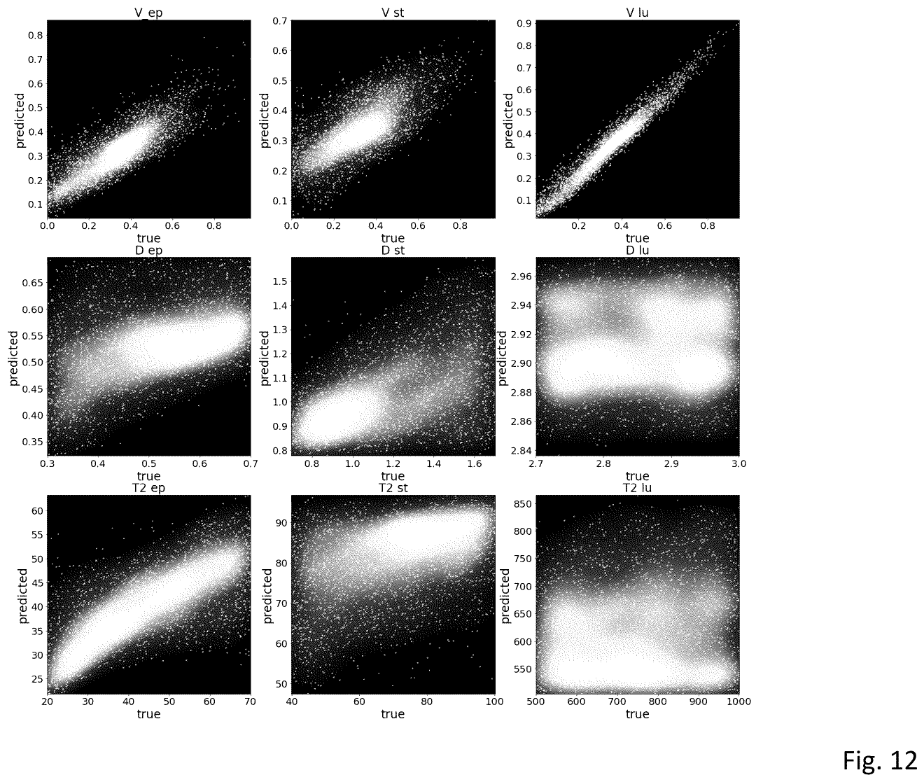

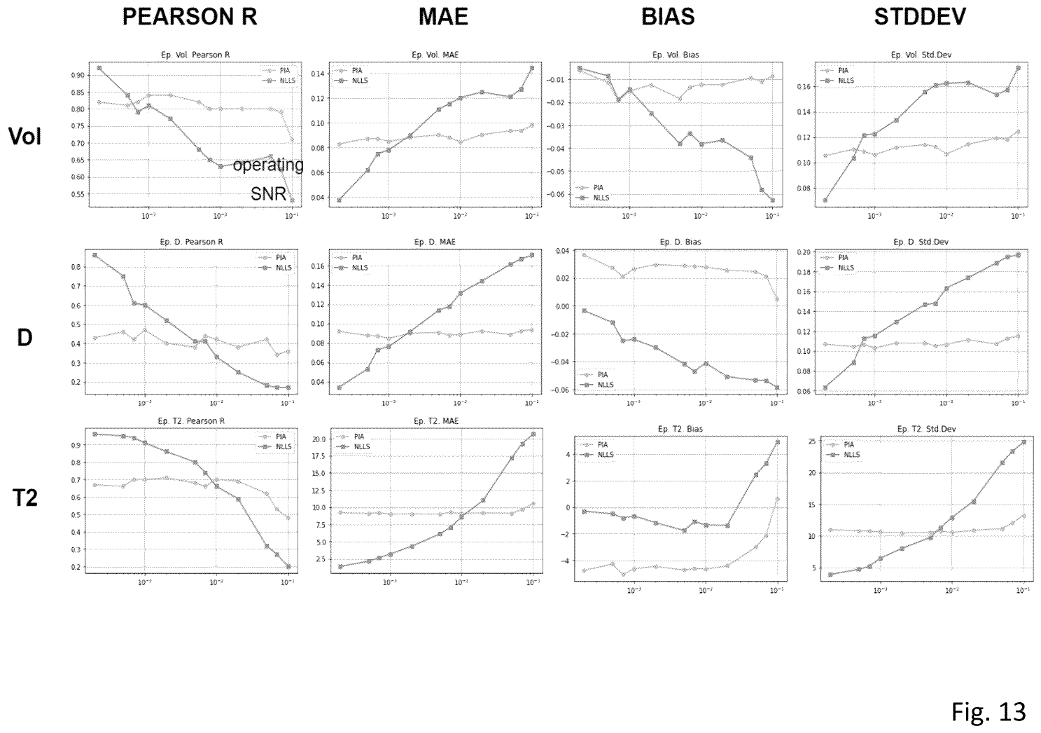

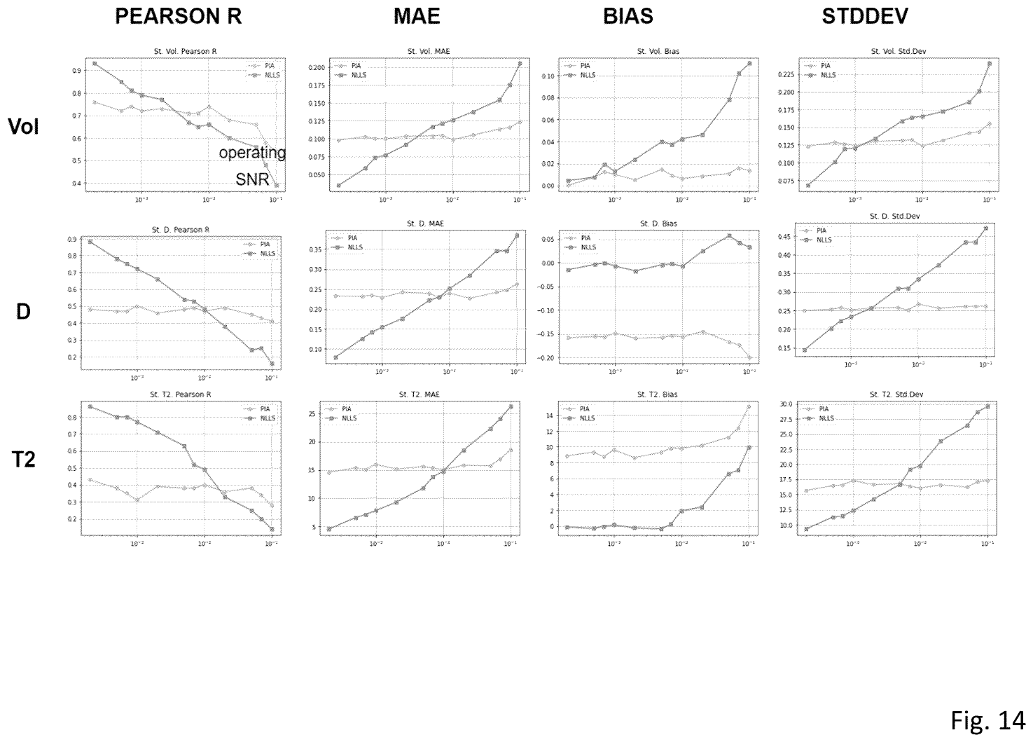

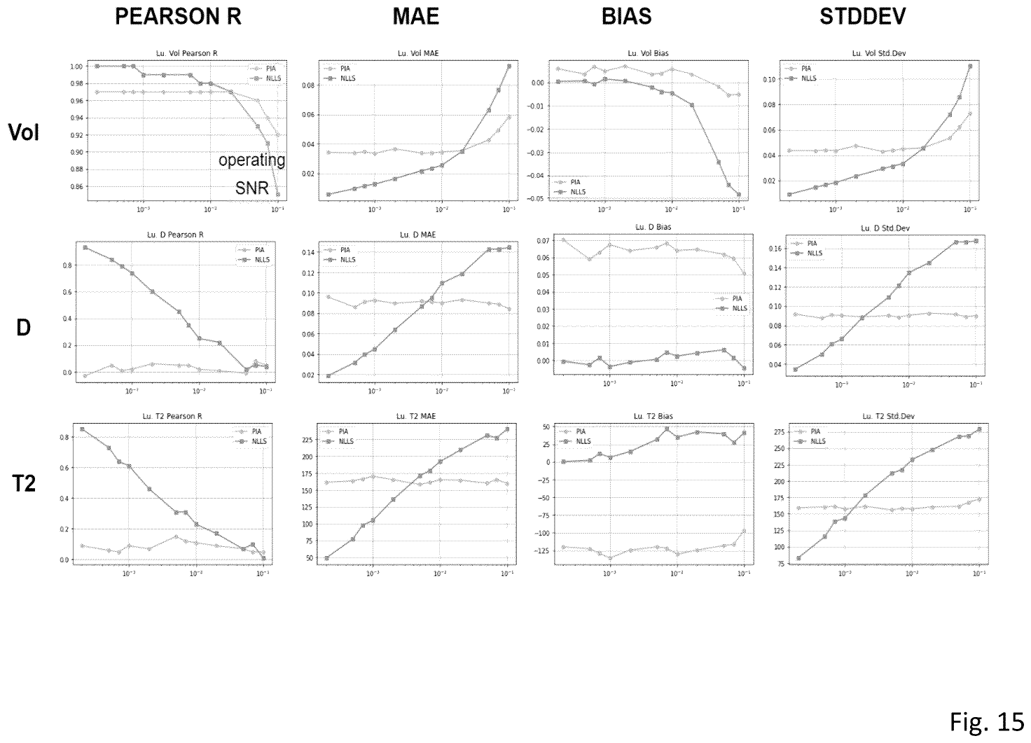

FIG. 11 depicts in-Silica scatter plots of the NLLS method at 20:1 SNR in accordance with an illustrative embodiment. The performance drop and pinning at the boundaries can be observed with the D and T2 measurements. FIG. 12 depicts in-Silica scatter plots of the PIA method at 20:1 SNR in accordance with an illustrative embodiment. FIGS. 13-15 demonstrate the change in performance with respect all metrics used in this study for the parameters corresponding to epithelium, stroma and lumen compartments. Specifically, FIG. 13 depicts the change in performance for the two methods, NLLS (squares) vs PIA (circles), on all tissue parameters (vol, D, T2) of the epithelium compartment, as a function of SNR in accordance with an illustrative embodiment. FIG. 14 depicts the change in performance for the two methods, NLLS (squares) vs PIA (circles), on all tissue parameters (vol, D, T2) of the stroma compartment, as a function of SNR in accordance with an illustrative embodiment. FIG. 15 depicts the change in performance for the two methods, NLLS (squares) vs PIA (circles), on all tissue parameters (vol, D, T2) of the lumen compartment, as a function of SNR in accordance with an illustrative embodiment.

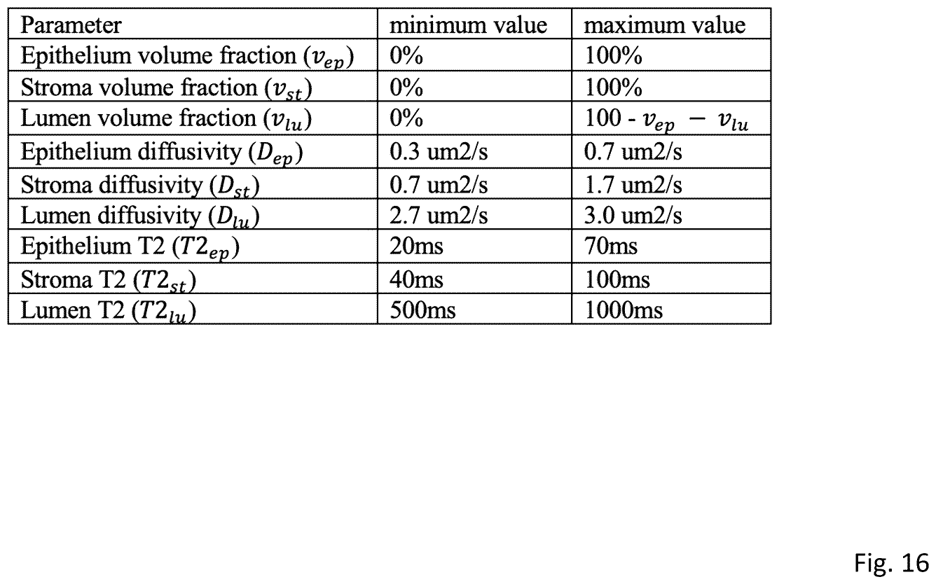

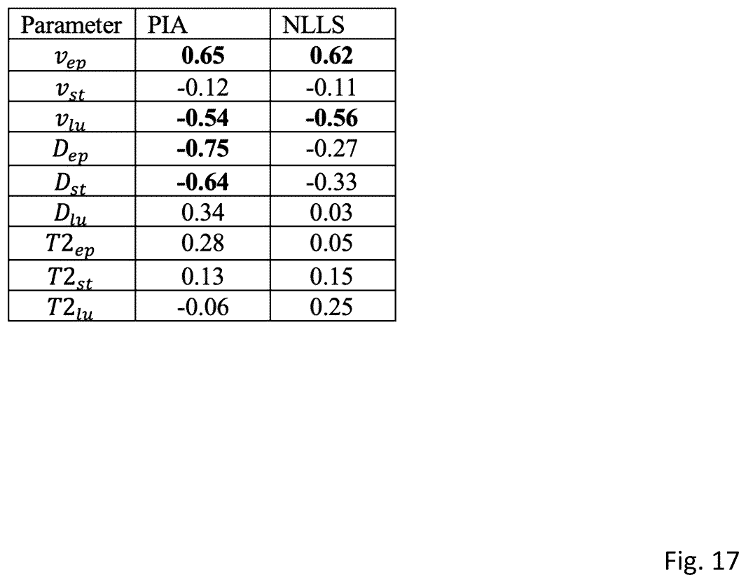

To conduct the study, the inventors used parameter ranges that were previously observed. FIG. 16 is a table that includes details for all parameter ranges considered in the experiments in accordance with an illustrative embodiment. FIG. 17 is a table that presents the Pearson Correlation coefficients tissue parameter measurements of PIA and NLLS methods, with respect to cancer aggressiveness, measured with the Gleason Score in accordance with an illustrative embodiment. In FIG. 17, statistically significant correlations are marked with bold face (p<0.01).

Additional information regarding the experimental setup and basis for the conducted experiments is included below. As discussed, the proposed system introduces a new paradigm that integrates the strengths of physics model-based and deep learning-based methods, while overcoming their respective weaknesses, including the need for large amounts of annotated data. To estimate the volume fractions (v) of epithelium, stroma, and lumen, as well as their diffusivities (D) and T2 relaxation times, used in physics model-based solutions, the inventors developed physics-informed autoencoders. This model treats these parameters not as unknowns of an equation set but as latent variables of an autoencoder.

The tissue composition estimation using PIA has been reimagined, transforming the problem from a least-squares fit into a robust deep learning framework. The PIA model includes a trainable multi-head neural network encoder and a fixed, physics-informed decoder. The encoder is a critical part of the PIA, including a multi-head deep neural network. The design of this neural network is focused on yielding accurate parameter estimates for each compartment, including volume fractions, T2, and ADC. The intricate architecture ensures that the underlying physics of the physics-model is integrated into the learning process, thereby creating a well-informed and robust encoding mechanism. The input to the encoder are multidimensional MRI signal measurements for each voxel. The first few layers of the encoder extract an embedding (e) from these measurements. This embedding is then fed to its multiple heads, each of which are deep neural networks, estimating a different parameter set, namely v, D and T2 for the 3 compartments: epithelium, stroma and lumen. e=encoder(S)

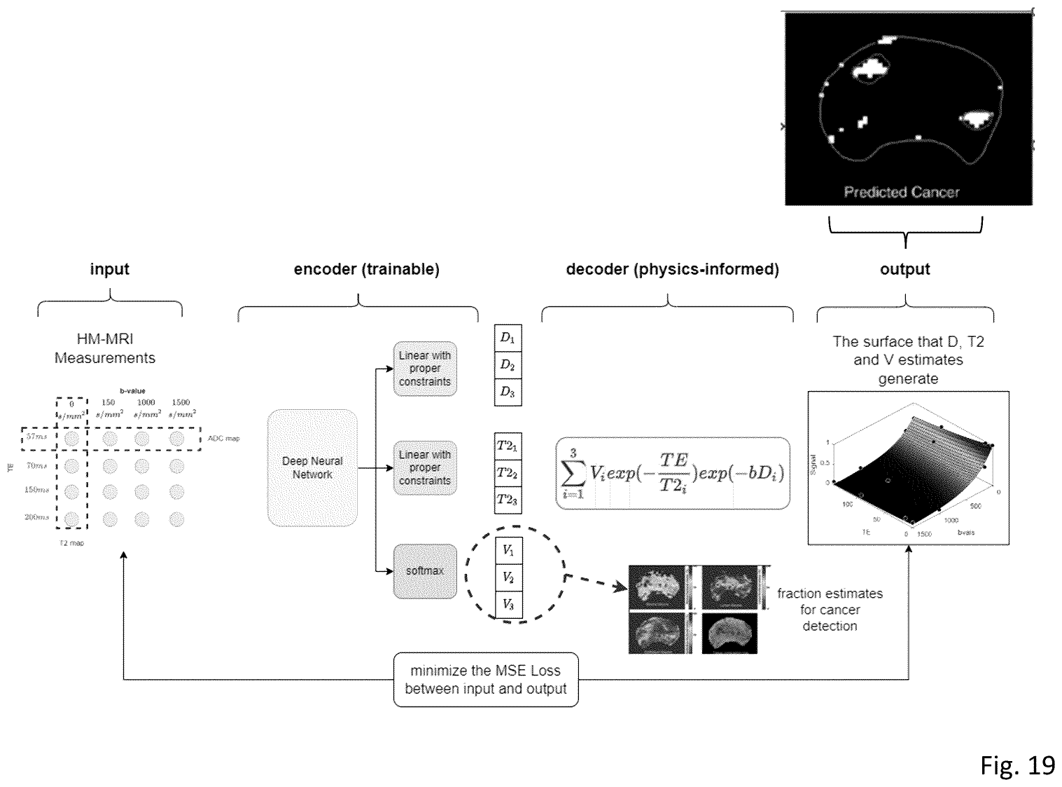

The decoder is uniquely set as nontrainable within the PIA architecture. Instead of learning from data, it employs a predefined analytical function that generates theoretical MRI signal values at the output. This aspect preserves the direct relationship between the encoded parameters and their corresponding theoretical values, ensuring that the deep learning model remains rooted in established physical laws, such as Equation 1, where gradient strength (b) and echo time (TE) are the imaging parameters used in acquiring the signal S. The overall flow of the model is presented in FIG. 19.

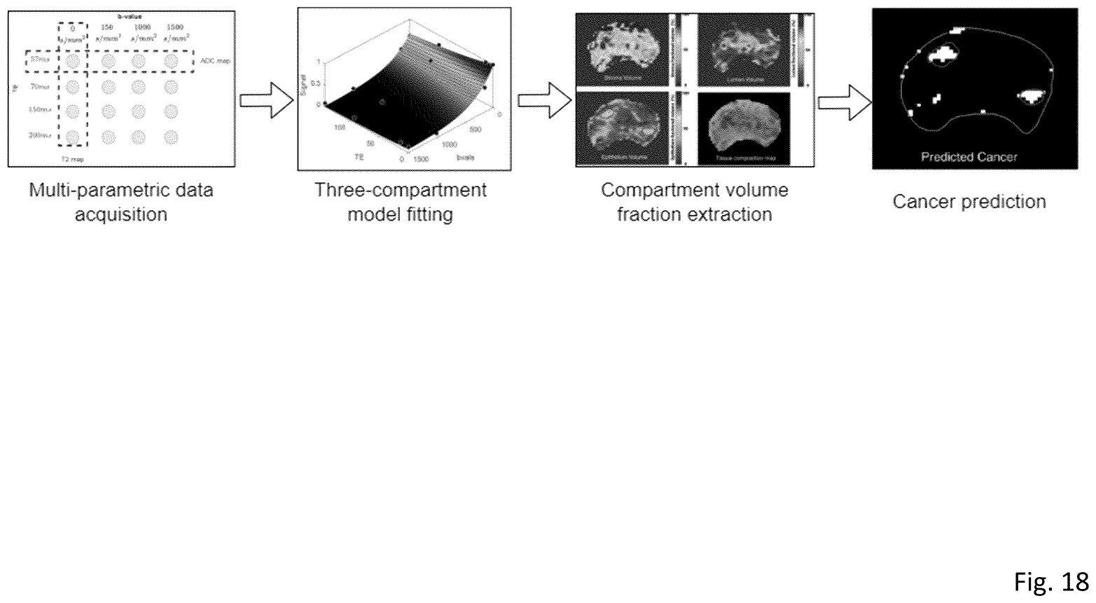

FIG. 18 is an overview of hybrid multidimensional MRI (HM-MRI) in accordance with an illustrative embodiment. The system obtains images of interest that are composed of image voxels. The images can be received directly from an MRI or other imaging machine, or stored in a memory. As shown in FIG. 18, the system collects a 4×4 matrix of signals as a function of ‘b’ and ‘TE’ for each image voxel in the image(s). The system then performs three-compartment model fitting a b/TE space, as shown in the second block of FIG. 18. This fit yields volume fractions of stroma, lumen, and epithelium in each voxel of the image(s). The system uses this tissue composition to generate and provide a cancer risk map that can be presented on a computer display and/or printed and provided to the patient such that he/she can visualize his/her cancer risk prediction.

FIG. 19 shows an embodiment of a physics-informed autoencoder for predicting cancer risk in accordance with an illustrative embodiment. As shown, the system receives HM-MRI measurements (e.g., in the form of images and/or raw data) as an input and uses an encoder to process the measurements. A decoder is used to generate an output, which includes a mapping that depicts regions of cancer risk based on the analysis.

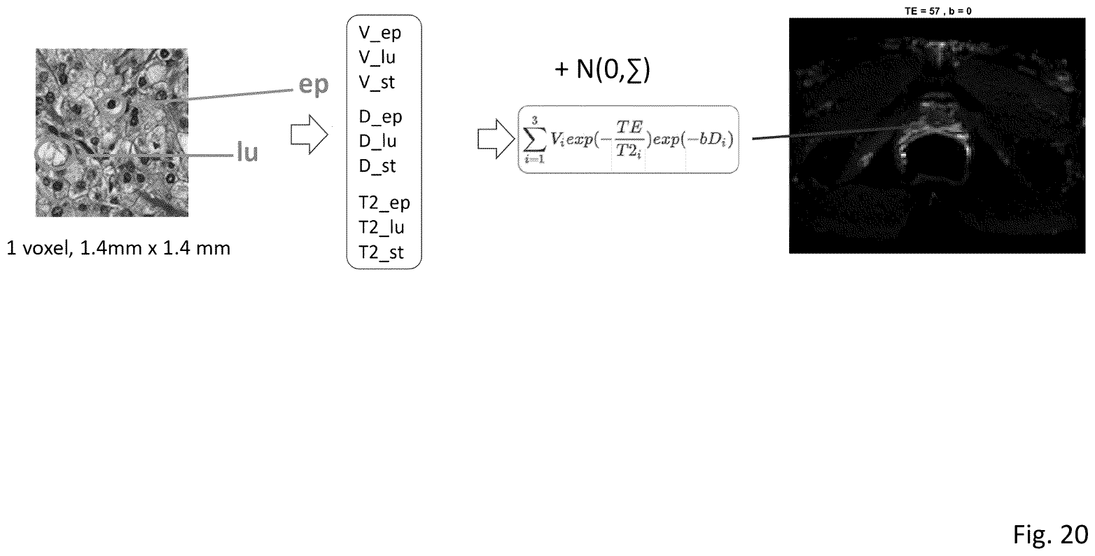

FIG. 20 depicts operations performed by an experimental setup of the proposed system in accordance with an illustrative embodiment. A voxel of an MRI image of prostate tissue is received by the system (e.g., directly from the MRI machine or from a database or other memory location). The voxel is 1.4 mm×1.4 mm in this embodiment. There are three major components within prostate tissue, lumen, epithelium, and stroma, and these can be identified from the voxel. Each of the three tissue components has different cell density and structure properties, such as volume fraction (V), diffusivity (D), and relaxation time (T2), which are identified by the system. These properties give rise to different MRI signal intensities. The signal model therefore exemplifies the evolution of the signal as a function of ‘b’ and ‘TE.’

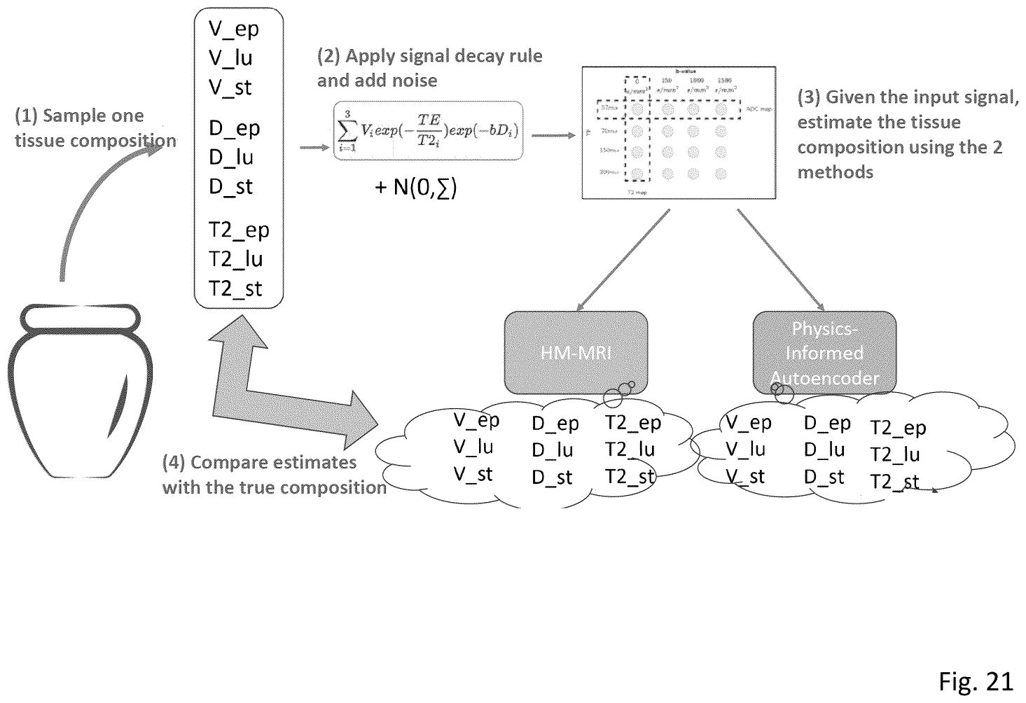

FIG. 21 depicts additional operations performed by an experimental setup of the proposed system in accordance with an illustrative embodiment. Using the experimental setups, it was determined that both HM-MRI and the proposed PIA detect dense epithelium on cancerous regions. However, PIA is able to detect that the epithelium diffusivity is lower on cancerous regions, while HM-MRI is unable to perform such detection. Also, as discussed herein, PIA performs neural network-based operations that performs estimations significantly faster than HM-MRI, which is a non-linear function fitting algorithm.

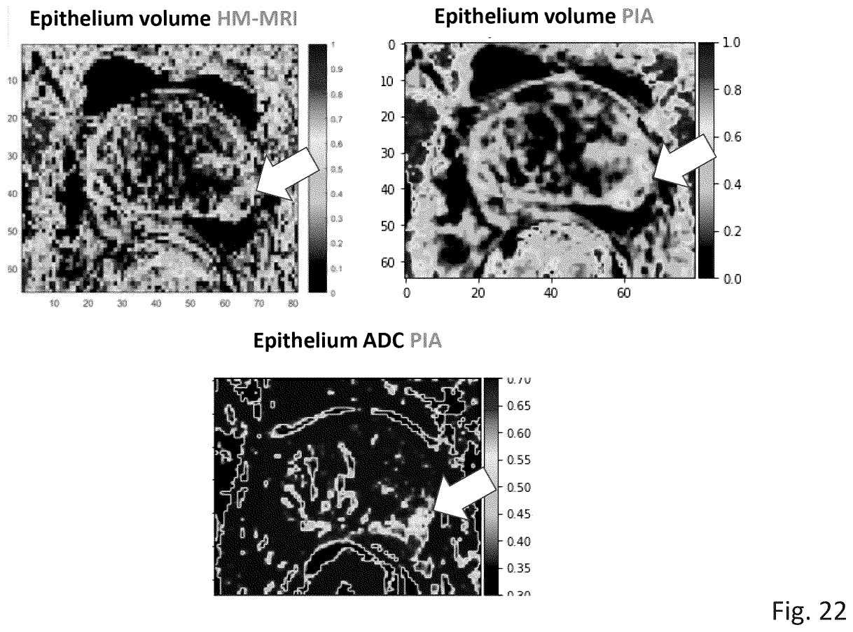

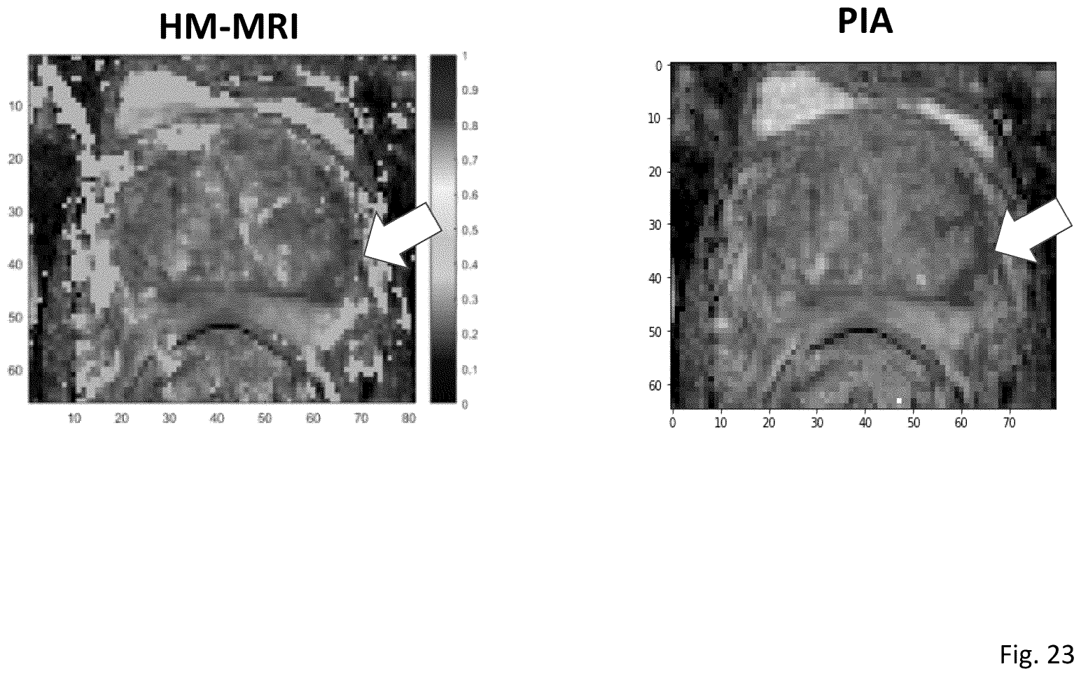

FIG. 22 depicts an epithelium volume obtained by HM-MRI, an epithelium volume map obtained by PIA, and an epithelium ADC map obtained by PIA in accordance with an illustrative embodiment. As indicated by the arrows, one cancer was missed by the HM-MRI analysis and identified using PIA. The PIA shows a larger region with denser epithelium and it is known that lower ADC for epithelium indicates dense rapidly growing cells. FIG. 23 depicts another example in which a cancer was missed by HM-MRI but identified using PIA in accordance with an illustrative embodiment. Using HM-MRI, the ADC map shows a faint area with restricted diffusion. However, using PIA the predicted cancer map clearly shows cancerous tissue.

In view of the methods and systems described above, the inventors also considered how prostate specific antigen (PSA) and PSA density (PSAD) have played a part in prostate cancer (PCa) diagnosis. An elevated PSA level may be caused by PCa but can also be caused by other conditions, including an enlarged prostate (benign prostatic hyperplasia) and inflammation (prostatitis). Therefore, PSA has low specificity and low positive predictive value for PCa diagnosis. Even correcting for prostate size, PSAD is inadequate for PCa screening. As a result, magnetic resonance imaging (MRI) is increasingly being used for PCa diagnosis.

It has been found that prostate specific antigen (PSA) and PSA density (PSAD) are inadequate for PCa screening. To improve screening, the inventors have developed a system to combine PSA and MRI measures from Hybrid Multi-dimensional MRI (HM-MRI) to improved PCa diagnosis. Specifically, blood PSA level, prostate volume from mpMRI and tissue volumes (epithelium, lumen) from HM-MRI were used to measure PSADₗᵤₘₑₙ and PSADₑₚᵢₜₕₑₗᵢᵤₘ normalized by tissue type (nPSAD). The results indicate that nPSADₑₚᵢₜₕₑₗᵢᵤₘ is significantly lower and nPSADₗᵤₘₑₙ is significantly higher in cancer patients compared to benign subjects. The diagnostic accuracy of nPSAD to detect subjects with PCa, was significantly higher than conventional PSAD, and further improved by combining nPSAD with tissue composition measures from HM-MRI. As a result, a new cancer biomarker has been identified that combines PSA (blood-based biomarker) with tissue composition from HM-MRI. nPSADₗᵤₘₑₙ and nPSADₑₚᵢₜₕₑₗᵢᵤₘ improve PCa diagnosis. These new biomarkers may signal effects of PCa on normal prostate and may indicate cancer aggressiveness. The new biomarkers and their development are described in more detail below.

PSA is produced in prostate epithelial cells and stored in a lumen (prostatic fluid) which has the highest concentration of PSA. The onset of cancer leads to breakdown of the barriers (loss of basal cell layer, increased leakiness, loss of tight junctions) between the glandular lumen and the capillaries, allowing more PSA to enter the circulation. As discussed above, the inventors have developed a way of measuring volume fractions of epithelium, stroma, and lumen non-invasively using Hybrid Multidimensional MRI (HM-MRI). Thus, one can specifically measure the volumes of tissue components that produce, secrete, and store PSA, and combine PSA and MRI measures for improved PCa diagnosis. The inventors conducted a study and determined that PSA density normalized by epithelial, stromal, and luminal fractional volume (nPSAD) from HM-MRI provides a useful independent biomarker that improves prostate cancer (PCa) diagnosis in comparison to conventional PSAD. As a result, introduced herein is a new cancer biomarker that combines PSA (blood-based biomarker) with tissue composition from HM-MRI. These normalized PSAD (nPSAD for lumen and epithelium) improve PCa diagnosis.

Conventional biomarkers such as PSAD are not significantly (p=0.14) different in men with cancer (0.19±0.11 ng/ml2) compared to benign subjects (0.15±0.08 ng/ml2). Using HM-MRI (old technique), PCa subjects had significantly higher epithelium (31.6±7.2 vs 17.9±5.2%, p<0.001) and reduced lumen (27.4±9.0 vs 40.4±9.8%, p<0.001) than benign subjects. Using the techniques described herein, it was found that nPSADₑₚᵢₜₕₑₗᵢᵤₘ (PSA produced per unit volume of cells producing them) in men with PCa (0.60±0.27 ng/ml2) is significantly (p=0.03) lower than benign subjects (0.92±0.57 ng/ml2). Additionally, it was found that nPSADₗᵤₘₑₙ (PSA per unit volume of ductal space) in men with PCa (0.81±0.66 ng/ml2) is significantly (p=0.01) higher than benign subjects (0.38±0.22 ng/ml2).

The diagnostic accuracy (AUC) of nPSAD to detect subjects with PCa, was significantly higher than conventional PSAD (0.61), with nPSADₗᵤₘₑₙ showing the best performance (0.76) followed by nPSADₑₚᵢₜₕₑₗᵢᵤₘ (0.64). Combining tissue composition with nPSAD measures from HM-MRI further improves performance (AUC=0.97).

It was found that even though PSA is produced by both benign epithelial and cancer cells, the level of expression on a per cell basis in PCa is known to be significantly lower by previous immunohistochemical studies, which is evidenced by lower nPSADₑₚᵢₜₕₑₗᵢᵤₘᵢₙ subjects with PCa than subjects with no PCa.

A decrease in the average luminal space in the prostate is correlated with an increased PSA in the blood. Normalizing PSAD with tissue measures allows one to detect the effect of PCa on secretion of PSA by the entire prostate. The effect of the cancer on nPSADₗᵤₘₑₙ and nPSADₑₚᵢₜₕₑₗᵢᵤₘ is too large to be explained by the change in the overall volume in the prostate of lumen and epithelium caused by the cancer. The volumes of the cancers studied and the differences between volume fractions of lumen and epithelium in cancer vs. benign tissue cannot produce a doubling (in the case of nPSADₗᵤₘₑₙ) or even a 50% increase (in the case of nPSADₑₚᵢₜₕₑₗᵢᵤₘ) measured in this study.

These results suggest that changes in the secretion of PSA in men with cancer cannot be explained by a linear combination of epithelium or lumen in cancer plus benign tissue. Rather the results suggest that the change in epithelium and lumen in the cancer has a systemic effect on the entire prostate. This is likely due to secretion of factors by the cancer. Based on this, the inventors hypothesized that the new biomarker presented here provides independent information regarding cancer aggressiveness. This information can be combined with measures of tissue composition from HM-MRI to improve diagnostic accuracy, as demonstrated by the large increase in AUC achieved by combining nPSAD with tissue composition from HM-MRI.

Based on the test results, the inventors have developed systems and methods to combine blood based biomarkers (PSA) and MRI measures from Hybrid Multi-dimensional MRI (HM-MRI) to improved prostate cancer (PCa) diagnosis. A new cancer biomarker is introduced that combines PSA (blood-based biomarker) with tissue composition from HM-MRI. As an example, the new biomarkers can be nPSADₗᵤₘₑₙ and nPSADₑₚᵢₜₕₑₗᵢᵤₘ. These new biomarkers signal effects of PCa on normal prostate and help to indicate presence of cancer and its aggressiveness. In one embodiment, prostate tissue compositions (fractional volumes of stroma, epithelium, and lumen) are calculated using a three-compartment model fit to HM-MRI data. Average fractional volumes over the entire prostate including cancers are then measured with HM-MRI for each subject to calculate normalized PSAD using the formula:

$$nPSAD\_tissue = PSA / ( prostate volume \times average fractional volume of tissue type ) \qquad Eq . 6$$

In the above-discussed study, patients with suspected PCa underwent 3T multi-parametric MRI (mpMRI) along with HM-MRI (TE=57, 75, 150, 200 ms, b-values=0, 150, 750, 1500 s/mm²) followed by biopsy. Clinical information: PSA level and prostate volume from mpMRI were used to measure PSAD. Prostate tissue composition (fractional volumes of stroma, epithelium, and lumen) was calculated using a three-compartment model fit to HM-MRI data similar to the previous studies. Average fractional volumes over the entire prostate including cancers (if any) were measured with HM-MRI for each subject to calculate nPSAD using Equation 1. PSAD and nPSAD results were compared for subjects with benign conditions vs. clinically significant cancers using t-test, and diagnostic performance was determined by ROC analysis.

Results and specifics of the study are as follows: 20 benign and 19 cancer (10 Gleason 3+4, 3 Gleason 4+3, 4 Gleason 4+4, 2 Gleason 4+5) subjects were included. PSAD was only nominally (p=0.14) higher in men with cancer (0.19±0.11 ng/ml) compared to benign subjects (0.15±0.08 ng/ml). PCa subjects had significantly higher epithelium (31.6±7.2 vs 17.9±5.2%, p<0.001) and reduced lumen (27.4±9.0 vs 40.4±9.8%, p<0.001) than benign subjects. Also, nPSADₑₚᵢₜₕₑₗᵢᵤₘ (PSA produced per unit volume of cells producing them) in men with PCa (0.60±0.27 ng/ml²) is significantly (p=0.03) lower than benign subjects (0.92±0.57 ng/ml²). Also, nPSADₗᵤₘₑₙ (PSA per unit volume of ductal space) in men with PCa (0.81±0.66 ng/ml²) is significantly (p=0.01) higher than benign subjects (0.38±0.22 ng/ml²). The diagnostic accuracy (AUC) of nPSAD to detect subjects with PCa, was significantly higher than conventional PSAD (0.61), with nPSADₗᵤₘₑₙ showing the best performance (0.76) followed by nPSADₑₚᵢₜₕₑₗᵢᵤₘ (0.64). Combining tissue composition with nPSAD measures from HM-MRI further improves performance (AUC=0.97).

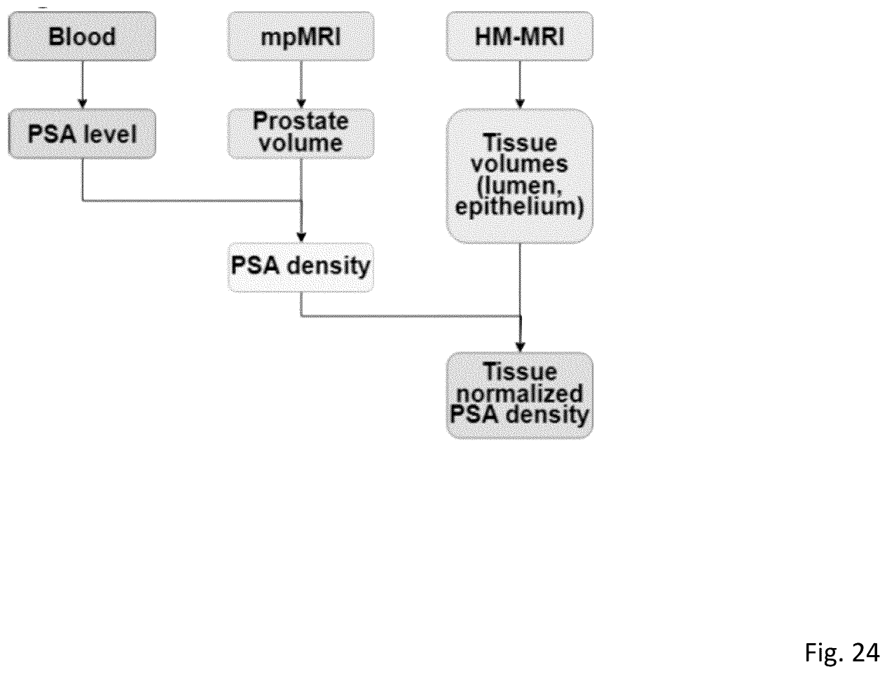

FIG. 24 depicts operations used to calculate tissue normalized PSA density in accordance with an illustrative embodiment. As shown, PSA level is determined from blood of a patient, prostate volume is determined by mpMRI, and tissue volumes (lumen, epithelium) are determined by HM-MRI. The PSA level and prostate volume are used to determine PSA density, and the PSA density is used along with the tissue volumes to determine tissue normalized PSA density, which is a biomarker that can be used to determine the likelihood of cancer being present.

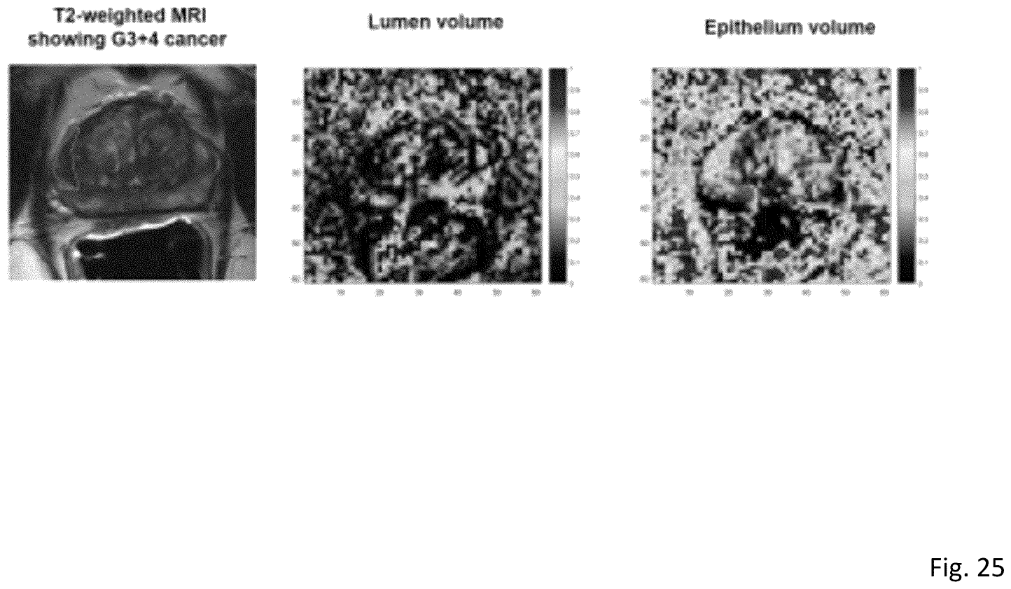

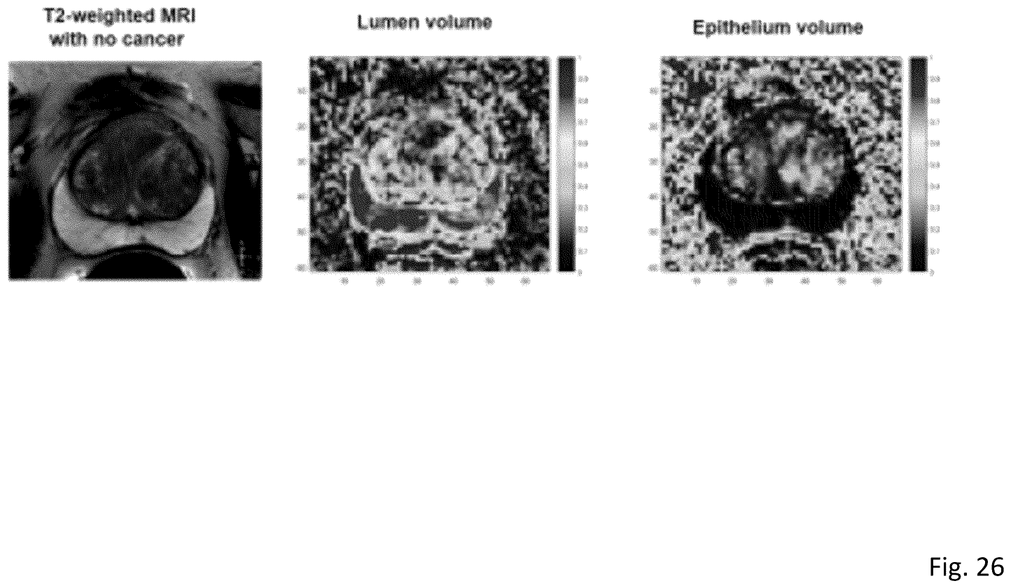

FIG. 25 depicts results of a patient with PSA of 10.1 ng/ml and prostate volume of 68.1 cc with Gleason 3+4 cancer in the right mid lateral region of the prostate in accordance with an illustrative embodiment. The cancer region shows increased epithelium and reduced lumen compared to surrounding benign issue. PSAD was 0.14 ng/ml and normalized PSAD: nPSADₑₚᵢₜₕₑₗᵢᵤₘ was 0.53 ng/ml² and nPSADₗᵤₘₑₙ was 0.65 ng/ml². FIG. 26 depicts a patient with PSA of 11.1 ng/ml and prostate volume of 96.8 cc with no cancer in accordance with an illustrative embodiment. As shown, PSAD was 0.11 ng/ml and normalized PSAD: nPSADₑₚᵢₜₕₑₗᵢᵤₘ was 0.81 ng/ml² and nPSADₗᵤₘₑₙ was 0.25 ng/ml².

Even though PSA is produced by both benign epithelial and cancer cells, the level of expression on a per cell basis in PCa is known to be significantly lower by previous immunohistochemical studies, which is evidenced by lower nPSAD in subjects with PCa than subjects with no PCa. A decrease in the average luminal space in the prostate is correlated with an increase PSA in the blood. Normalizing PSAD with tissue measures allowed the inventors to detect the effect of PCa on secretion of PSA by the entire prostate. The effect of the cancer on nPSADₗᵤₘₑₙ and nPSADₑₚᵢₜₕₑₗᵢᵤₘ is too large to be explained by the change in the overall volume in the prostate of lumen and epithelium caused by the cancer. The volumes of the cancers studied and the differences between volume fractions of lumen and epithelium in cancer vs. benign tissue cannot produce a doubling (in the case of nPSADₗᵤₘₑₙ) or even a 50% increase (in the case of nPSADₑₚᵢₜₕₑₗᵢᵤₘ) measured in this study. This suggests that changes in the secretion of PSA in men with cancer cannot be explained by a linear combination of epithelium or lumen in cancer plus benign tissue. Rather the results suggest that the change in epithelium and lumen in the cancer has a systemic effect on the entire prostate. This is likely due to secretion of factors by the cancer. Based on this—the inventors hypothesized that the new biomarker presented here provides independent information regarding cancer aggressiveness. This information can be combined with measures of tissue composition from HM-MRI to improve diagnostic accuracy, as demonstrated by the large increase in AUC achieved by combining nPSAD with tissue composition from HM-MRI.

Thus, the new biomarker presented herein provides independent information regarding cancer aggressiveness. This information can be combined with measures of tissue composition from HM-MRI to improve diagnostic accuracy, as demonstrated by the large increase in AUC achieved by combining nPSAD with tissue composition from HM-MRI.

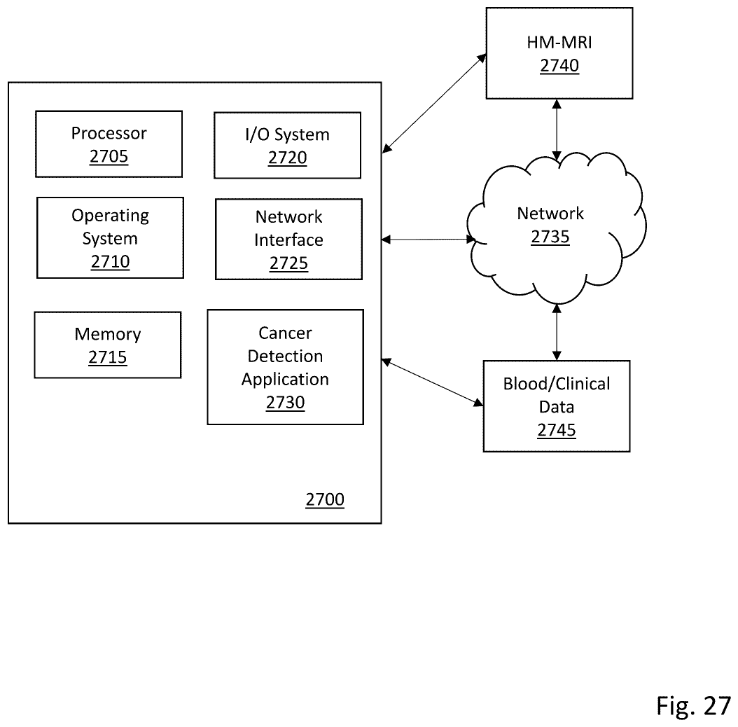

In an illustrative embodiment, any of the operations described herein can be performed by a computing system that includes a processor, a memory, a user interface, a transceiver (e.g., a receiver and transmitter), etc. The operations described herein can be implemented as computer-readable instructions that are stored in the memory. Upon execution of the computer-readable instructions by the processor, the computing system performs the various operations. The computing system can be in the form of a dedicated computing system, a personal computing device (e.g., smartphone), a laptop computer, desktop computer, etc. FIG. 27 depicts a computing system 2700 for performing image analysis and cancer risk assessment in accordance with an illustrative embodiment.

The computing system 2700 includes a processor 2705, an operating system 2710, a memory 2715, an input/output (I/O) system 2720, a network interface 2725, and a cancer detection application 2730. In alternative embodiments, the computing system 2700 may include fewer, additional, and/or different components. The components of the computing system 2700 communicate with one another via one or more buses or any other interconnect system. As discussed, the computing system 2700 can be any type of computing system (e.g., smartphone, tablet, laptop, desktop, etc.), including a dedicated standalone computing system that is designed to control and/or perform analysis in support of an MRI system. The computing system 2700 is connected to a network 2735, which can be any type of computing network. Either directly or through the network 2735, the computing system 2700 is also connected to an HM-MRI machine 2740 and to blood/clinical data 2745. The HM-MRI machine 2740 can be used to capture images (e.g., images containing voxels) of the prostate or other tissue and provide the images to the computing system 2700 for analysis. Alternatively, data corresponding to the images can be provided to the computing system 2700. The blood/clinical data 2745 can include information obtained from a blood draw of the patient, which is used to determine PSA of the patient. The determined PSA can then be used to determine a normalized PSAD as described herein.

The processor 2705 can be in electrical communication with and used to control any of the system components described herein. For example, the processor 2705 can be used to execute the cancer detection application 2730, control the HM-MRI machine 2740, process data from the HM-MRI machine, process the blood/clinical data 2745, generate output maps and/or reports, etc. The processor 2705 can be any type of computer processor known in the art and can include a plurality of processors and/or a plurality of processing cores. The processor 2705 can include a controller, a microcontroller, an audio processor, a graphics processing unit, a hardware accelerator, a digital signal processor, etc. Additionally, the processor 2705 may be implemented as a complex instruction set computer processor, a reduced instruction set computer processor, an x86 instruction set computer processor, etc. The processor 2705 is used to run the operating system 2710, which can be any type of operating system.

The operating system 2710 is stored in the memory 2715, which is also used to store programs, received images and image data, network and communications data, peripheral component data, the cancer detection application 2730, and other operating instructions. The memory 2715 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 2715 can be in the cloud to provide cloud storage for the system. Similarly, in one embodiment, any of the computing components described herein (e.g., the processor 2705, etc.) can be implemented in the cloud such that the system can be run and controlled through cloud computing.

The I/O system 2720 is the framework which enables users and peripheral devices to interact with the computing system 2700. The I/O system 2720 can also include one or more speakers, one or more microphones, a keyboard, a mouse, a display, one or more buttons or other controls, etc. that allow the user to interact with and control the computing system 2700. The I/O system 2720 can also include a printer to print maps that identify cancerous regions within tissue, cancer risk assessments, etc. The I/O system 2720 also includes circuitry and a bus structure to interface with peripheral computing devices such as the imaging system, power sources, universal service bus (USB) devices, data acquisition cards, peripheral component interconnect express (PCIe) devices, serial advanced technology attachment (SATA) devices, high-definition multimedia interface (HDMI) devices, proprietary connection devices, etc.

The network interface 2725 includes transceiver circuitry (e.g., a transmitter and a receiver) that allows the computing system 2700 to transmit and receive data to/from other devices such as remote computing systems, servers, websites, imaging systems, etc. The network interface 2725 enables communication through the network 2735, which can be one or more communication networks. The network 2735 can include a cable network, a fiber network, a cellular network, a wi-fi network, a landline telephone network, a microwave network, a satellite network, etc. The network interface 2725 also includes circuitry to allow device-to-device communication such as Bluetooth® communication.

The cancer detection application 2730 can include software and algorithms in the form of computer-readable instructions which, upon execution by the processor 2705, performs any of the various operations described herein such as controlling the HM-MRI machine to generate images of a prostate, to receive captured images and/or image data, analyzing images to obtain volume fractions of stoma, lumen, and epithelium, analyze the blood/clinical data 2745 to determine PSA, analyze images to determine tissue composition, analyze images to determine volume estimates, ADC estimates, and T2 estimates, determine a location of cancer based on volume estimates, ADC estimates, and/or T2 estimates, generate, using a printer, a printed map that identifies locations that may be cancerous, determine a severity of the identified cancerous locations, perform any other operations of the physics-informed autoencoder, etc. The cancer detection application 2730 can utilize the processor 2705 and/or the memory 2715 as discussed above. In an alternative implementation, the cancer detection application 2730 can be remote or independent from the computing device 2700, but in communication therewith.

Various acronyms are used herein, and these acronyms have the following meaning: ADC=apparent diffusion coefficient, AUC=area under the receiver operating characteristic curve, CSPCa=clinically significant PCa, HM-MRI=hybrid multidimensional MRI, ICC=intraclass correlation coefficient, MAE=mean absolute error, NLLS=nonlinear least squares, PCa =prostate cancer, PIA=Physics-Informed Autoencoder, PI-RADS=Prostate Imaging Reporting and Data System, ROI=region of interest, SNR=signal-to-noise ratio.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system for assessing cancer, the system comprising:
a memory configured to store one or more images of tissue of a patient, wherein the one or more images originate from a magnetic resonance imaging (MRI) machine;
a processor operatively coupled to the memory and configured to:
determine a composition of the tissue;
determine, based at least in part on the composition of the tissue, an apparent diffusion constant of the tissue;
identify a region of cancer based at least in part on the apparent diffusion constant; and
generate a map that identifies the region of cancer in the tissue.

2. The system of claim 1, wherein the composition of the tissue includes a fractional volume of stroma in the tissue.

3. The system of claim 1, wherein the composition of the tissue includes a fractional volume of lumen in the tissue.

4. The system of claim 1, wherein the composition of the tissue includes a fractional volume of epithelium in the tissue.

5. The system of claim 1, wherein the one or more images comprise a plurality of voxels, and wherein the processor applies a 3-compartment diffusion-relaxation signal model to each voxel in the plurality of voxels.

6. The system of claim 1, wherein the processor determines, based on the composition of the tissue, one or more relaxation times (T2) of the tissue, wherein the region of cancer is identified based at least in part on the one or more relaxation times.

7. The system of claim 1, wherein the processor determines, based on the composition of the tissue, a volume of the tissue, and wherein the region of cancer is identified based at least in part on the volume of the tissue.

8. The system of claim 1, wherein the processor uses an encoder to determine the apparent diffusion constant of the tissue.

9. The system of claim 1, wherein the apparent diffusion constant is determined with respect to an epithelium portion of the tissue, a lumen portion of the tissue, and a stroma portion of the tissue.

10. The system of claim 1, wherein the processor determines an echo time and a b-value of the tissue from the one or more images, and wherein the composition of the tissue is determined based at least in part on the echo time and the b-value.

11. The system of claim 1, wherein the tissue comprises prostate tissue, and the processor is configured to determine a normalized prostate specific antigen (PSA) density of the prostate tissue, and wherein the region of cancer is identified based on the normalized PSA density, which acts as a biomarker.

12. The system of claim 11, wherein the tissue comprises prostate tissue, and wherein the processor determines a first normalized PSA density of an epithelial portion of the prostate tissue and a second normalized PSA density of a lumen portion of the prostate tissue.

13. The system of claim 11, wherein the processor determines the normalized PSA density based on prostate volume, tissue volumes within the prostate, and a PSA density of the prostate tissue.

14. A method of assessing cancer risk, the method comprising:
receiving, by a memory of a computing system, one or more images of tissue, wherein the one or more images originate from a magnetic resonance imaging (MRI) machine;
determining, by a processor of the computing system, a composition of the tissue;
determining, by the processor and based at least in part on the composition of the tissue, an apparent diffusion constant of the tissue;
identifying, by the processor, a region of cancer based at least in part on the apparent diffusion constant; and
generating, by the processor, a map that identifies the region of cancer in the tissue.

15. The method of claim 14, wherein determining the composition of the tissue includes determining a fractional volume of stroma in the tissue, determining a fractional volume of lumen in the tissue, and determining a fractional volume of epithelium in the tissue.

16. The method of claim 14, further comprising determining, by the processor and based on the composition of the tissue, one or more relaxation times (T2) of the tissue, wherein the region of cancer is identified based at least in part on the one or more relaxation times.

17. The method of claim 14, determining the apparent diffusion constant comprises determining a first apparent diffusion constant with respect to an epithelium portion of the tissue, determining a second apparent diffusion constant with respect to a lumen portion of the tissue, and determining a third apparent diffusion constant with respect to a stroma portion of the tissue.

18. The method of claim 14, further comprising determining, by the processor, an echo time and a b-value of the tissue from the one or more images, wherein the composition of the tissue is determined based at least in part on the echo time and the b-value.

19. The method of claim 14, wherein the tissue comprises prostate tissue, and further comprising determining, by the processor a normalized prostate specific antigen (PSA) density of the prostate tissue, and wherein the region of cancer is identified based on the normalized PSA density, which acts as a biomarker.

20. The method of claim 19, wherein the processor determines the normalized PSA density based on prostate volume, tissue volumes within the prostate, and a PSA density of the prostate tissue.
